(19) United States
(12) Patent Application Publication
(10) Pub. No.: US 2024/0199743 A1
(43) Pub. Date: Jun. 20, 2024

(54) COMPOSITIONS AND METHODS FOR TREATING CHRONIC ACTIVE WHITE MATTER LESIONS / RADIOLOGICALLY ISOLATED SYNDROME

(71) Applicant: BIOGEN MA INC., Cambridge, MA (US)

(72) Inventor: Shibeshih Mitiku BELACHEW, Cambridge, MA (US)

(21) Appl. No.: 18/286,721

(22) PCT Filed: Apr. 13, 2022
(86) PCT No.: PCT/US22/24694

§ 371 Date: Oct. 12, 2023

Related U.S. Application Data
(60) Provisional application No. 63/174,399, filed on Apr. 13, 2021.
(60) Provisional application No. 63/320,655, filed on Mar. 16, 2022.

(30) Foreign Application Priority Data
Apr. 13, 2021 (FR) FR2103793

(51) Int. Cl. C07K 16/28; A61K 9/00; A61K 39/00; A61P 25/28

(52) U.S. Cl. CPC C07K 16/2842; A61K 9/0019; A61P 25/28; A61K 2039/505; A61K 2039/545

(57) ABSTRACT

Provided herein are methods for administering disease-modifying antibody therapies to asymptomatic and/or early-stage multiple sclerosis patients, including Radiologically Isolated Syndrome patients, based on the identification and/or co-localization of slowly expanding lesions and paramagnetic rim lesions in magnetic resonance images from said patients.

### CROSS-REFERENCE TO RELATED APPLICATIONS

This application claims the benefit of priority to U.S. Provisional Application No. 63/174,399, filed on Apr. 13, 2021, U.S. Provisional Application No. 63/320,655, filed on Mar. 16, 2022, French Provisional Application No. FR2103793, filed on Apr. 13, 2021, and International Application No. PCT/US2022/024450 filed on Apr. 12, 2022.

### BACKGROUND

Multiple sclerosis (MS) is a chronic, inflammatory, demyelinating disease of the central nervous system typically characterized by focal lesions in the white matter, and generally presents with a spectrum of clinical phenotypes ranging from relapsing (RMS) and relapsing-remitting (RRMS) to increasingly progressive forms. The phenotypic categorization of MS into relapsing and primary progressive multiple sclerosis (PPMS) and other forms resulted from definitions put forth in 1996 by the US National Multiple Sclerosis Society (NMSS) Advisory Committee on Clinical Trials in Multiple Sclerosis. Notably, however, the distinctions drawn were not made on the basis of a fundamental, well-established, and well-understood scientific characterization of the pathophysiology of the various forms. To the contrary, at the time they were proposed the committee acknowledged that the phenotypic descriptors were consensus subjective views of experts in the field and were not supported by objective biological findings.

As clinicians have more recently come to appreciate, the differences between these various clinical phenotypes are relative rather than absolute. For example, while some evidence suggests that primary progressive multiple sclerosis (PPMS) represents a distinct, non-inflammatory or at least less inflammatory pathologic form of MS than secondary progressive multiple sclerosis (SPMS), abundant clinical, imaging, and genetic data suggest that PPMS is a part of the spectrum of progressive MS phenotypes, and analyses of natural history cohorts demonstrate that worsening proceeds at a similar rate in both SPMS and PPMS. Lublin, Fred D., et al. Defining the Clinical Course of Multiple Sclerosis: The 2013 Revisions. Neurology. 2014 Jul. 15; 83(3): 278-86. Accordingly, there is growing awareness in the field that there is substantial overlap of the subtypes with regard to clinical subtypes and likely pathophysiology.

Given this emerging understanding as well as the inherent subjectivity of clinical phenotyping in general, attention has gradually turned to various pathophysiological characterizations based on the underlying biological presentation of the disease. There is a common view that the underlying pathology of MS involves both inflammation and neurodegeneration. Lublin et al. Neurology The 2013 clinical course descriptors for multiple sclerosis: A clarification 2020:94:1-5. doi.10.1212/WNL.0000000000009636. The relationship between the clinical evolution of the disease and these mechanisms is complex, however, and in need of further characterization. Id.

One facet of this relates to the prevalence and expansion of white matter lesions based on magnetic resonance imaging (MRI). MRI is the most sensitive imaging technique for detecting MS lesions in vivo, and lesion load measurements based on conventional T2-weighted MRI are widely used to monitor treatment effects in therapeutic trials. Notably, however, there is only a modest correlation between the lesion load on conventional MRI and the clinical disability of patients with MS, a phenomenon referred to as clinicoradiologic dissociation. Seewan et al., Arch Neurol. 2009; 66(5):601-609. doi: 10.1001/archneurol.2009.57. As such, more accurate and informative radiological methods are still clearly needed to inform appropriate treatment decisions for patients.

Individuals with radiologically isolated syndrome (RIS) have incidental MRI abnormalities suggestive of MS. Recent studies using susceptibility-based imaging have shown that a subgroup of chronic active white matter lesions (CAWMLs) have a rim of paramagnetic susceptibility-associated signal loss at the lesion edge, the paramagnetic rim sign (PRS), that is associated with the presence of iron inside phagocytes, which indicates chronic, active demyelination. Suthiphosuwan et al., JAMA Neuro. Paramagnetic Rim Sign in Radiologically Isolated Syndrome Mar. 9, 2020 doi: 10.1001/jamaneurol.20200124. As such, these patients may indeed benefit from the more aggressive disease-modifying treatment options normally reserved for relapsing and progressive forms of MS. Unfortunately, however, given the historical emphasis on subjective clinical phenotypes rather than pathophysiology in the determination and approval of available treatments, the RIS patient population in particular has few treatment options and the disease is typically left to exacerbate until clinical manifestations become more apparent.

Tysabri® (natalizumab) is an anti-very late antigen (VLA)-4 humanized monoclonal IgG4 antibody that inhibits the migration of lymphocytes throughout the blood-brain barrier by blocking VLA-4 interactions with vascular cell adhesion molecules (VCAM)-1 and reducing inflammatory lesions. Natalizumab is a biotherapeutic approved for treating relapsing and progressive forms of multiple sclerosis. Under U.S. clinical practice, natalizumab is only used in patients who have had at least one relapse event, as determined by their clinician, while in Europe natalizumab treatment requires the diagnosis of at least one relapse event along with the identification of at least one acute lesion (typically defined as an increase in lesion load/size in a T2 or a T1 gadolinium-enhanced lesion on MRI). To date, in view of attendant treatment risks as well as the long-established reliance on clinical phenotypes to drive treatment decisions, there has been no consensus on its potential use in earlier stage disease.

### SUMMARY

The present disclosure provides methods for treating and/or reducing chronic white matter lesion activity (CWMLA), also referred to as chronic lesion activity (CLA), in patients in need thereof, including Radiologically Isolated Syndrome (RIS) patients, with appropriate disease-modifying therapies, e.g. anti-VLA-4 antibodies. In particular, the present invention demonstrates that certain presentations of CWMLA visible with specific magnetic resonance imaging techniques correlate with disease progression, and thus patients having this confluence of radiological markers can be effectively treated with more aggressive disease-modifying antibody therapies even in the absence of the clinical manifestations (e.g., relapses) conventionally used to justify such therapies. Accordingly, new methods of treating earlier-stage and/or asymptomatic (e.g., pre-first episode of relapse) patients such as RIS patients based on the presence of new radiological characterizations of CWMLA are described and exemplified herein.

In one aspect, the disclosure provides methods for treating Radiologically Isolated Syndrome (RIS) in a patient in need thereof comprising administering a therapeutically effective amount of a disease-modifying antibody therapy to said patient, wherein said patient has CWMLA as defined by at least one phase rim lesion (PRL) in at least one susceptibility-weighted magnetic resonance image (MRI). In some embodiments, the patient has CWMLA as defined by at least one slowly expanding lesion (SEL). In some embodiments, the patient has CWMLA as defined by at least one SEL that is detected using single time-point non-contrast T1- and T2-weighted MRI.

In some embodiments, the patient has CWMLA as defined by at least one SEL that co-localizes with at least one PRL, or vice-versa. In some embodiments, the at least one SEL is detected using single time-point non-contrast T1- and T2-weighted MRI.

In some embodiments, the SEL is detected using a machine-learning based classifier that discriminates acute from chronic MS lesions, and/or SEL from non-SEL, using unenhanced T1/T2 information from a single MRI scan. Advantageously, then, a patient suspected of having RIS, or a patient previously identified as having RIS and at further risk of developing MS, may be referred for an MRI scan of the brain at a single time point, and without agent contrast. The scan may then be input into the classifier algorithm, which may identify and distinguish between acute and chronic lesions present on the brain scan, and/or between SEL and non-SEL. Based on that identification and distinction, an appropriate disease-modifying antibody therapy can be administered that is suitable to the particular patient and disease state.

In some embodiments, one or more features having predictive value with respect to the classification of a lesion as either acute or chronic and/or as SELs are utilized. In some embodiments, said features are selected from the group comprising or consisting of: features that quantify the first order intensity of the core region of a lesion as it appears on a T2-weighted scan image; features that quantify the amount of signals appearing as low-gray around the periphery of a lesion as it appears on a T1-weighted scan image; features that quantify the amount of high-gray signals that are present in the periphery and/or core of a lesion as it appears on a T1-weighted scan image; features that relate to the inhomogeneity present in the images; features that relate to the structure of the image, as relating to the presence of repeating patterns; and features that relate to the texture of the images.

In some embodiments, the disease-modifying antibody therapy is selected from natalizumab, BIIB107 and ocrelizumab. In some embodiments, the disease-modifying antibody therapy is an anti-VLA-4 antibody, e.g. natalizumab or BIIB107.

In some embodiments, the anti-VLA-4 antibody is natalizumab administered in a biphasic dosing regimen, wherein the biphasic regimen comprises an induction phase comprising administration of natalizumab once a month for about 10 to about 14 months followed by a chronic phase comprising administration of natalizumab once every 5, 6, 7, or 8 weeks. In some embodiments, the induction phase comprises administration of natalizumab once a month for about 10 months, about 11 months, about 12 months, about 13 months, about 14 months, or longer than about 14 months. In some embodiments, at least one phase of a biphasic dosing regimen comprises subcutaneous (SC) administration. In some embodiments, both the induction phase and chronic phase of a biphasic dosing regimen comprises SC injection. In some embodiments, the induction phase of a biphasic dosing regimen comprises SC injection. In some embodiments, the chronic phase of a biphasic dosing regimen comprises SC injection. In some embodiments, surprisingly, the SC dosing and amount of natalizumab can be consistent with IV dosing. In some embodiments, the therapeutically effective amount administered during the induction phase and the chronic phase are the same, and the therapeutically effective amount is between 250-450 mg (e.g. 250 mg, 300 mg, 350 mg, 400 mg, or 450 mg), more preferably about 300 mg, still more preferably 300 mg. In some embodiments, the therapeutically effective amount administered SC during the chronic phase is between 300-500 mg (e.g., 300 mg, 350 mg, 400 mg, 450 mg, or 500 mg). In some embodiments, the therapeutically effective amount is between about 250-about 450 mg (e.g., about 250 mg, about 300 mg, about 350 mg, about 400 mg, or about 450 mg), more preferably about 300 mg, still more preferably 300 mg. In some embodiments, the therapeutically effective amount administered SC during the chronic phase is between about 300-about 500 mg (e.g., about 300 mg, about 350 mg, about 400 mg, about 450 mg, or about 500 mg), more preferably about 300 mg, still more preferably 300 mg.

In some embodiments, the anti-VLA-4 antibody is natalizumab administered in a chronic dose regimen, wherein the chronic dosing regimen comprises administration of natalizumab at a fixed interval of every 4 weeks. In some embodiments, the chronic dosing regimen is a fixed, non-weight based amount of natalizumab. In some embodiments, a therapeutically effective amount of natalizumab is between about 250-450 mg, or about 300 mg. In some embodiments, a therapeutically effective amount of natalizumab is a fixed, non-weight based dose of 300 mg. In further embodiments, the chronic dosing regimen is every 4 weeks for a period of about 4 months, about 5 months, about 6 months, about 7 months, about 8 months, about 9 months, about 10 months, about 11 months, about 12 months, or longer than about 12 months. In some embodiments, the chronic dosing regimen comprises SC injection. In some embodiments, the chronic dosing regimen comprises IV administration.

In some embodiments, treatment with anti-VLA-4 therapy is initiated when at least 1%, 5%, 10%, 15%, 20%, 25%, 30%, 35%, 40%, 45%, 50%, 55%, 60%, 65%, 70%, or 75% of the patient's total T2 hyperintense lesion volume and/or number is identified as PRL. In some embodiments, treatment with anti-VLA-4 therapy is initiated when at least 1%, 5%, 10%, 15%, 20%, 25%, 30%, 35%, 40%, 45%, 50%, 55%, 60%, 65%, 70%, or 75% of the patient's total T2 hyperintense lesion volume and/or number is identified as SEL. In some embodiments, treatment with anti-VLA-4 therapy is initiated when at least 5%, 10%, 15%, 20%, 25%, 30%, 35%, 40%, 45%, 50%, 55%, 60%, 65%, 70%, or 75% of the patient's SELs co-localize with their PRLs. In some embodiments, treatment with anti-VLA-4 therapy is initiated when at least 5%, 10%, 15%, 20%, 25%, 30%, 35%, 40%, 45%, 50%, 55%, 60%, 65%, 70%, or 75% of the patient's PRLs co-localize with their SELs.

In another aspect, the invention provides methods of reducing and/or treating chronic active white matter lesions in an asymptomatic and/or early-stage MS patient (e.g. having no diagnosed relapses, or fulfilling consensus diagnostic criteria, e.g. Thompson A J et al. Lancet Neurol. 2018; 17:162-173) comprising administering a therapeutically effective amount of a disease-modifying antibody therapy to said patient, wherein said patient has CWMLA as defined by at least one phase rim lesion (PRL) in at least one susceptibility-weighted magnetic resonance image (MRI). In some embodiments, the patient has CWMLA as defined by at least one slowly expanding lesion (SEL). In some embodiments, the patient has CWMLA as defined by at least one SEL that is detected using single time-point non-contrast T1- and T2-weighted MRI.

In some embodiments, the patient has CWMLA as defined by at least one SEL that co-localizes with at least one PRL, or vice-versa. In some embodiments, the at least one SEL is detected using single time-point non-contrast T1- and T2-weighted MRI.

In some embodiments, the SEL is detected using a machine-learning based classifier that discriminates acute from chronic MS lesions and/or SEL from non-SEL using unenhanced T1/T2 information from a single MRI scan. Advantageously, then, an asymptomatic and/or early stage patient suspected of having a brain ailment such as MS, or an asymptomatic and/or early stage patient at risk of developing MS, may be referred for an MRI scan of the brain at a single time point, and without agent contrast. The scan may then be input into the classifier algorithm, which may identify and distinguish between acute and chronic lesions present on the brain scan, and/or between SEL and non-SEL. Based on that identification and distinction, an appropriate disease-modifying antibody therapy can be administered that is suitable to the particular patient and disease state.

In some embodiments, one or more features having predictive value with respect to the classification of a lesion as either acute or chronic and/or as SELs are utilized. In some embodiments, said features are selected from the group comprising or consisting of: features that quantify the first order intensity of the core region of a lesion as it appears on a T2-weighted scan image; features that quantify the amount of signals appearing as low-gray around the periphery of a lesion as it appears on a T1-weighted scan image; features that quantify the amount of high-gray signals that are present in the periphery and/or core of a lesion as it appears on a T1-weighted scan image; features that relate to the inhomogeneity present in the images; features that relate to the structure of the image, as relating to the presence of repeating patterns; and features that relate to the texture of the images.

In some embodiments, the disease-modifying antibody therapy is selected from natalizumab, BIIB107 and ocrelizumab. In some embodiments, the disease-modifying antibody therapy is an anti-VLA-4 antibody, e.g. natalizumab or BIIB107. In some embodiments, the anti-VLA-4 antibody is natalizumab administered in a biphasic dosing regimen, wherein the biphasic regimen comprises an induction phase comprising administration of natalizumab once a month for about 10 to about 14 months, followed by a chronic phase comprising administration of natalizumab once every 5, 6, 7 or 8 weeks. In some embodiments, the induction phase comprises administration of natalizumab once a month for about 10 months, about 11 months, about 12 months, about 13 months, about 14 months, or longer than about 14 months. In some embodiments, at least one phase of a biphasic protocol comprises subcutaneous (SC) administration. In some embodiments, both the induction phase and chronic phase of a biphasic dosing regimen comprises SC injection. In some embodiments, the induction phase of a biphasic dosing regimen comprises SC injection. In some embodiments, the chronic phase of a biphasic dosing regimen comprises SC injection. In some embodiments, surprisingly, the SC dosing and amount of natalizumab can be consistent with IV dosing. In some embodiments, the therapeutically effective amount administered during the induction phase and the chronic phase are the same, and the therapeutically effective amount is between 250-450 mg (e.g. 250 mg, 300 mg, 350 mg, 400 mg, or 450 mg), more preferably about 300 mg, still more preferably 300 mg. In some embodiments, the therapeutically effective amount administered SC during the chronic phase is between 300-500 mg (e.g., 300 mg, 350 mg, 400 mg, 450 mg, or 500 mg). In some embodiments, the therapeutically effective amount is between about 250-about 450 mg (e.g., about 250 mg, about 300 mg, about 350 mg, about 400 mg, or about 450 mg), more preferably about 300 mg, still more preferably 300 mg. In some embodiments, the therapeutically effective amount administered SC during the chronic phase is between about 300-about 500 mg (e.g., about 300 mg, about 350 mg, about 400 mg, about 450 mg, or about 500 mg), more preferably about 300 mg, still more preferably 300 mg.

In some embodiments, the anti-VLA-4 antibody is natalizumab administered in a chronic dose regimen, wherein the chronic dosing regimen comprises administration of natalizumab at a fixed interval of every 4 weeks. In some embodiments, the chronic dosing regimen is a fixed, non-weight based amount of natalizumab. In some embodiments, a therapeutically effective amount of natalizumab is between about 250-450 mg, or about 300 mg. In some embodiments, a therapeutically effective amount of natalizumab is a fixed, non-weight based dose of 300 mg. In further embodiments, the chronic dosing regimen is every 4 weeks for a period of about 4 months, about 5 months, about 6 months, about 7 months, about 8 months, about 9 months, about 10 months, about 11 months, about 12 months, or longer than about 12 months. In some embodiments, the chronic dosing regimen comprises SC injection. In some embodiments, the chronic dosing regimen comprises IV administration.

In some embodiments, treatment with anti-VLA-4 therapy is initiated when at least 1%, 5%, 10%, 15%, 20%, 25%, 30%, 35%, 40%, 45%, 50%, 55%, 60%, 65%, 70%, or 75% of the patient's total T2 hyperintense lesion volume and/or number is identified as PRL. In some embodiments, treatment with anti-VLA-4 therapy is initiated when at least 1%, 5%, 10%, 15%, 20%, 25%, 30%, 35%, 40%, 45%, 50%, 55%, 60%, 65%, 70%, or 75% of the patient's total T2 hyperintense lesion volume and/or number is identified as SEL. In some embodiments, treatment with anti-VLA-4 therapy is initiated when at least 5%, 10%, 15%, 20%, 25%, 30%, 35%, 40%, 45%, 50%, 55%, 60%, 65%, 70%, or 75% of the patient's SELs co-localize with their PRLs. In some embodiments, treatment with anti-VLA-4 therapy is initiated when at least 5%, 10%, 15%, 20%, 25%, 30%, 35%, 40%, 45%, 50%, 55%, 60%, 65%, 70%, or 75% of the patient's PRLs co-localize with their SELs.

In another aspect, methods for reducing and/or treating chronic white matter lesion activity in an asymptomatic and/or early-stage MS patient (e.g. having no diagnosed relapse events) are provided comprising a) identifying at least one phase rim lesion (PRL) in at least one susceptibility-weighted magnetic resonance image from a patient known or suspected of having chronic active white matter lesions, b) identifying at one slowly-expanding lesion (SEL) in at least one T1-weighted/T2-weighted MRI from said patient; c) determining if the at least one PRL co-localizes with the at least one SEL in said patient, and/or vice-versa, and d) in the event of co-localization initiating treatment with a disease-modifying antibody therapy. In some embodiments, the at least one SEL is detected using single time-point non-contrast T1- and T2-weighted MRI. In some embodiments, the disease-modifying antibody therapy is selected from natalizumab, BIIB107 and ocrelizumab. In some embodiments, the disease-modifying antibody therapy is an anti-VLA-4 antibody, e.g. natalizumab or BIIB107.

In some embodiments, the SEL is detected using a machine-learning based classifier that discriminate acute from chronic MS lesions and/or SEL from non-SEL using unenhanced T1/T2 information from a single MRI scan. Advantageously, then, an asymptomatic and/or early stage patient suspected of having a brain ailment such as MS, or an asymptomatic and/or early stage patient at risk of developing MS, may be referred for an MRI scan of the brain at a single time point, and without agent contrast. The scan may then be input into the classifier algorithm, which may identify and distinguish between acute and chronic lesions present on the brain scan, and/or between SEL and non-SEL. Based on that identification and distinction, an appropriate disease-modifying antibody therapy can be administered that is suitable to the particular patient and disease state.

In some embodiments, one or more features having predictive value with respect to the classification of a lesion as either acute or chronic and/or as SELs are utilized. In some embodiments, said features are selected from the group comprising or consisting of: features that quantify the first order intensity of the core region of a lesion as it appears on a T2-weighted scan image; features that quantify the amount of signals appearing as low-gray around the periphery of a lesion as it appears on a T1-weighted scan image; features that quantify the amount of high-gray signals that are present in the periphery and/or core of a lesion as it appears on a T1-weighted scan image; features that relate to the inhomogeneity present in the images; features that relate to the structure of the image, as relating to the presence of repeating patterns; and features that relate to the texture of the images.

In some embodiments, the method further comprises administering to said patient a therapeutically effective amount of natalizumab in a biphasic dosing regimen, wherein the biphasic dosing regimen comprises an induction phase comprising administration of the anti-VLA-4 antibody once every 2 weeks, about once very 2 weeks, once every 4 weeks, about once every 4 weeks, once every 30 days, about once every 30 days, once a month or about once a month for at least 6 months, for at least 8 months, for at least 10 months, or for at least 12 months, followed by a chronic phase comprising administration of the anti-VLA-4 antibody once every 5 to 10 weeks, or once every 5, 6, 7 or 8 weeks. In some embodiments, the induction phase is from 6 to 18 months, from 8 to 16 months, from 10 to 14 months, or is 11 months, is 12 months or is 13 months. In some embodiments, the induction phase is 12 months, and the chronic phase comprises administration of natalizumab every 5 weeks, about every 5 weeks, every 6 weeks, about every 6 weeks, every 7 weeks or about every 7 weeks. In some embodiments, the induction phase is 12 months and the chronic phase comprises administration of natalizumab every 6 weeks.

In some embodiments, treatment with anti-VLA-4 therapy is initiated when at least 5%, 10%, 15%, 20%, 25%, 30%, 35%, 40%, 45%, 50%, 55%, 60%, 65%, 70%, or 75% of the patient's SELs co-localize with their PRLs. In some embodiments, treatment with anti-VLA-4 therapy is initiated when at least 5%, 10%, 15%, 20%, 25%, 30%, 35%, 40%, 45%, 50%, 55%, 60%, 65%, 70%, or 75% of the patient's PRLs co-localize with their SELs.

### BRIEF DESCRIPTION OF THE DRAWINGS

FIG. 1: Association between CWMLA or whole brain volume loss and composite disability progression in the placebo arm. Change from baseline to week 108 in T1LV in (A) SELs, (B) non-SELs, and (C) CNT2 lesions was significantly associated with composite disability progression in SPMS patients. No difference in percentage change from baseline to week 108 in (D) whole brain volume was observed in SPMS patients with composite disability progression compared with those with no progression. Composite progression was confirmed at 24 weeks and end of study on one or more of the EDSS, Timed 25-Foot Walk, or 9-Hole Peg Test. In these box-and-whisker representations, the box spans the interquartile range, the median is marked by the horizontal line inside the box, and the whiskers are the two lines outside the box that extend to the highest and lowest observations. p values by Van Elteren test; stratified by baseline EDSS score (≤5.5 or ≥6.0) and baseline T2 lesion volume category based on tertiles (≤6908.79 mm³, >6908.79-18818.49 mm³, and >18818.49 mm³). CNT2=chronic nonenhancing T2; CWMLA=chronic white matter lesion activity; EDSS=Expanded Disability Status Scale; SEL=slowly expanding lesion; SPMS=secondary progressive multiple sclerosis; T1LV=T1-hypointense lesion volume.

FIG. 2: Association between CWMLA and EDSS progression. Increase from baseline to week 108 in T1LV within (A) SELs and (C) CNT2 lesions but not (B) non-SELs was associated with EDSS progression in SPMS patients treated with placebo. (However, a consistent directional trend was observed in non-SELs.) EDSS progression was confirmed at 24 weeks and end of study. In these box-and-whisker representations, the box spans the interquartile range, the median is marked by the horizontal line inside the box, and the whiskers are the two lines outside the box that extend to the highest and lowest observations. p values by Van Elteren test; stratified by baseline EDSS score (≤5.5 or ≥6.0) and by baseline T2 lesion volume category based on tertiles (≤6908.79 mm³, >6908.79-18818.49 mm³, and >18818.49 mm³). CNT2=chronic nonenhancing T2; CWMLA=chronic white matter lesion activity; EDSS=Expanded Disability Status Scale; SEL=slowly expanding lesion; SPMS=secondary progressive multiple sclerosis; T1LV=T1-hypointense lesion volume.

FIG. 3: Association between CWMLA and 9HPT progression. Change from baseline to week 108 in T1LV within (A) SELs and (C) CNT2 lesions but not (B) non-SELs was associated with 9HPT progression in SPMS patients treated with placebo. (However, a consistent directional trend was observed in non-SELs.) 9HPT progression was confirmed at 24 weeks and end of study. In these box-and-whisker representations, the box spans the interquartile range, the median is marked by the horizontal line inside the box, and the whiskers are the two lines outside the box that extend to the highest and lowest observations. p values by Van Elteren test; stratified by baseline EDSS score (≤5.5 or ≥6.0) and by baseline T2 lesion volume category based on tertiles (≤6908.79 mm³, >6908.79-18818.49 mm³, and >18818.49 mm³). 9HPT=9-Hole Peg Test; CNT2=chronic nonenhancing T2; CWMLA=chronic white matter lesion activity; EDSS=Expanded Disability Status Scale; SEL=slowly expanding lesion; SPMS=secondary progressive multiple sclerosis; T1LV=T1-hypointense lesion volume.

FIG. 4: Association between CWMLA and T25FW progression. Change in T1LV from baseline to week 108 within (B) non-SELs and (C) CNT2 lesions but not (A) SELs was associated with T25FW progression in SPMS patients treated with placebo. (However, a consistent directional trend was observed in SELs.) T25FW progression was confirmed at 24 weeks and end of study. In these box-and-whisker representations, the box spans the interquartile range, the median is marked by the horizontal line inside the box, and the whiskers are the two lines outside the box that extend to the highest and lowest observations. p values by Van Elteren test; stratified by baseline EDSS score (≤5.5 or ≥6.0) and by baseline T2 lesion volume category based on tertiles (≤6908.79 mm³, >6908.79-18818.49 mm³, and >18818.49 mm³). CNT2=chronic nonenhancing T2; CWMLA=chronic white matter lesion activity; EDSS=Expanded Disability Status Scale; SEL=slowly expanding lesion; SPMS=secondary progressive multiple sclerosis; T1LV=T1-hypointense lesion volume; T25FW=Timed 25-Foot Walk.

FIG. 5: Association between CWMLA and composite disability progression in the absence of AWMLA. CWMLA in (B) non-SELs and (C) CNT2 lesions but not (A) SELs remained associated with composite disability progression in the absence of AWMLA in SPMS patients treated with placebo. (However a consistent directional trend was observed in SELs.) Absence of acute lesion activity was defined as no baseline and postbaseline T1 gadolinium-enhancing and no postbaseline new/enlarging T2 lesions. Composite progression was confirmed at 24 weeks and end of study on one or more of the EDSS, Timed 25-Foot Walk, or 9-Hole Peg Test. In these box-and-whisker representations, the box spans the interquartile range, the median is marked by the horizontal line inside the box, and the whiskers are the two lines outside the box that extend to the highest and lowest observations. p values by Van Elteren test; stratified by baseline EDSS score (≤5.5 or ≥6.0) and baseline T2 lesion volume category based on tertiles (≤6908.79 mm³, >6908.79-18818.49 mm³, and >18818.49 mm³). AWMLA=acute white matter lesion activity, also referred to as acute lesion activity (ALA); CNT2=chronic nonenhancing T2; CWMLA=chronic white matter lesion activity; EDSS=Expanded Disability Status Scale; SEL=slowly expanding lesion; SPMS=secondary progressive multiple sclerosis; T1LV=T1-hypointense lesion volume.

FIG. 6: Prevalence of SELs and frequency distribution of SEL severity in the presence versus absence of AWMLA. SEL (A) number, (B) absolute volume, and (C) relative volume (percentage of baseline nonenhancing T2LV) was greater in SPMS patients treated with placebo who had AWMLA compared to those with no AWMLA. (D) The frequency distribution of patients by range of SEL prevalence indicates that patients with AWMLA had a greater percentage of their total T2 lesion burden identified as SELs compared with patients with no AWMLA. In these box-and-whisker representations, the box spans the interquartile range, the median is marked by the horizontal line inside the box, and the whiskers are the two lines outside the box that extend to the highest and lowest observations. No acute lesion activity was defined as no baseline or postbaseline Gd+ T1 lesions and no postbaseline new/enlarging T2 lesions in weeks 24, 48, 72, 96, and 108. Acute lesions were defined as baseline Gd+ T1 lesions and postbaseline Gd+ T1 lesions and new/enlarging T2 lesions in weeks 24, 48, 72, 96, and 108. p values by Van Elteren test: stratified by baseline EDSS score (≤5.5 or ≥6.0) and baseline T2LV category based on tertiles (≤6908.79 mm³, >6908.79-18818.49 mm³, and >18818.49 mm³). AWMLA=acute white matter lesion activity; BL=baseline; EDSS=Expanded Disability Status Scale; Gd+=gadolinium enhancing; SEL=slowly expanding lesion; SPMS=secondary progressive multiple sclerosis; T2LV=T2-hyperintense lesion volume.

FIG. 7: Effect of natalizumab on SEL prevalence. Natalizumab reduced the (A) number, (B) absolute volume, and (C) relative volume (percentage of baseline nonenhancing T2LV) of SELs in SPMS patients. Box-and-whisker representations, the box spans the interquartile range, the median is marked by the horizontal line inside the box, and the whiskers are the two lines outside the box that extend to the highest and lowest observations. p values by Van Elteren test; stratified by baseline Expanded Disability Status Scale score (≤5.5 or ≥6.0) and baseline T2LV category based on tertiles (≤6908.79 mm³, >6908.79-18818.49 mm³, and >18818.49 mm³). SEL=slowly expanding lesion; SPMS=secondary progressive multiple sclerosis; T2LV=T2-hyperintense lesion volume; T2w=T2 weighted.

FIG. 8: Change in CWMLA with natalizumab versus placebo. Natalizumab reduced CWMLA as measured by both (A, B) absolute increase and (C, D) percentage increase in T1LV in SELs and non-SELs compared with placebo in SPMS patients. Distribution-free quantile confidence limits are displayed. p values by Van Elteren test; stratified by baseline EDSS score (≤5.5 or ≥6.0) and baseline T2 lesion volume category based on tertiles (≤6908.79 mm³, >6908.79-18818.49 mm³, and >18818.49 mm³). BL=baseline; CI=confidence interval; CWMLA=chronic white matter lesion activity; EDSS=Expanded Disability Status Scale; SPMS=secondary progressive multiple sclerosis; T1LV=T1-hypointense lesion volume.

FIGS. 9A-9E: Overlap of SELs and PRLs. SELs identified based on change within pre-existing lesion from screening to week 72, outlined on T1w images at (A) screening and (B) week 72. (C) Phase rim (PRL) annotations outlined on a co-registered frequency map at week 72. (D) T2-lesions associated with the PRLs, corresponding to the area within the rims in (C), overlaid on the FLAIR image at week 72. (E) Voxel-wise overlap between the SELs and T2-lesions associated with the PRLs (arrows denote voxels that overlap. Also shown are voxels only present in SEL, and voxels only in T2-lesions associated with PRLs).

FIG. 10: Correlation between number of SELs versus number of PRLs. Number of PRLs is depicted on the x-axis, and number of SELs is depicted on the y-axis.

FIGS. 11A-11B: Comparisons of lesions across types. PRL size with and without SEL co-localization (A), and SEL size with and without PRL co-localization (B).

FIGS. 12A-12B: Evolution of tissue damage within SEL/PRL lesions. Comparison of normalized magnetization transfer ratio (nMTR) trajectories, PRL with and without SEL colocalization (A) and SEL with and without PRL colocalization (B). In (A) nonPRL; PRL, SEL; PRL, non-SEL. In (B) Non-SEL; SEL, PRL; SEL, nonPRL. In (A) and (B), computed as weighted means over samples (PRL or SEL). Shaded areas represent 95% CI of mean.

FIGS. 13A-13B: Evolution of tissue damage within SEL/PRL lesions. Comparison of radial diffusivity trajectories. PRL with and without SEL colocalization (A) and SEL with and without PRL colocalization (B). In (A) nonPRL; PRL, SEL; PRL, non-SEL. In (B) non-SEL; SEL, PRL; SEL, nonPRL. In (A) and (B), computed as weighted means over samples (PRL or SEL). Shaded areas represent 95% CI of mean.

FIG. 14: Selection of two patches (one SEL, one non-SEL) extracted from chronic unenhancing MS lesions of a brain T2 MRI scan.

FIGS. 15A-15B: Non-SEL patch extracted from baseline T2 MRI scan showing the core and periphery regions. (A) unhighlighted, (B) core (solid line) and periphery (dashed line).

FIGS. 16A-16C: Illustrations of lesion volume matching between SEL and Non-SEL patches (top, SEL; bottom, non-SEL). Each of (A), (B), and (C) correspond to a volume-matched pair.

FIG. 17: Prevalence of each of the fifteen radiomic features selected for discriminating SEL from non-SEL patches, with abbreviations as follows: t1p: T1-weighted MRI pre-contrast (i.e., feature was extracted from non-contrast T1-weighted MRI image); t2w: T2-weighted MRI (i.e., feature was extracted from T2-weighted MRI image); core/periphery: specifies whether feature was computed within core or periphery region; glrlm: Gray-level Run-Length Matrix; glcm: Gray-level Cooccurence Matrix; glszm: Gray-level Size Zone Matrix. Notably, first-order statistics including the mean, median and 90th percentile of T1 intensities in the core of the patch were identified as relevant, consistent with prior studies reporting that SELs exhibit a higher degree of T1 hypo-intensity relative to non-SELs at baseline.

FIGS. 18A-18D: Confusion matrices showing the performance of the classification model for patch-level SEL versus non-SEL discrimination on the training, validation, and independent testing sets. (A) ADVANCE training set (balanced accuracy: 73.0%), (B) ADVANCE validation set (balanced accuracy: 66.8%), (C) ASCEND test set (balanced accuracy: 65.7%), (D) SYNERGY test set (balanced accuracy: 68.5%).

FIGS. 19A-19D: Confusion matrices showing the performance of the classification model for patch-level SEL versus non-SEL discrimination on the training, validation, and independent testing sets for volume-matched patches.

### DETAILED DESCRIPTION

Natalizumab, sold under the trade name TYSABRI® (BIOGEN®, MA), is an integrin receptor antagonist approved by the U.S. Food and Drug administration (FDA) for treatment of multiple sclerosis and Crohn's disease. The FDA approved standard dosing regimen is 300 milligrams (mg) infused intravenously over approximately one hour, every four weeks. Among the population of patients who have received natalizumab therapy, there is a small subpopulation of patients who have developed progressive multifocal leukoencephalopathy (PML) (Plavina, T. et al. Ann Neurol 2014; 76:802-12). Substantial efforts have been made to identify and minimize this risk, including the development of a wide range of patient monitoring and alternative dosing protocols. Nevertheless, in view of these risks as well as costs and other considerations, there is general reluctance in the field to use natalizumab or other more aggressive disease-modifying antibody therapies with earlier stage disease, including in particular in asymptomatic patients who have yet to exhibit any of the clinical manifestations of MS.

The availability of magnetic resonance imaging has led to an increase in the detection of abnormal brain findings even in cases when there are no outward symptoms. When the MRI findings are similar to those seen in MS patients, but the patient is asymptomatic of the typical physical or neurological symptoms associated with MS, e.g., relapses, this is known as radiologically isolated syndrome (RIS). Although there is a strong association between RIS and MS, an RIS diagnosis does not always progress to an MS diagnosis. Indeed, when followed over a two year period, only about one third of patients with RIS develop a neurological event and are diagnosed with MS, while one third develop a new finding on MRI without any symptoms, and the last third show no change. What is needed, then, are improved methods for identifying those RIS patients more likely to progress to MS, so that more effective treatment decisions can be made earlier in the disease process.

Chronic active lesions, also known as smoldering plaques, are a neuropathologic hallmark of chronic inflammation in multiple sclerosis (Elliott et al. Patterning chronic active demyelination in slowly expanding/evolving white matter MS legions. AJNR Am J Neuroradiol dx.doi.org/10.3174/ajnr.A6742). The chronic active lesions are generally surrounded by a rim of activated microglia and/or macrophages that may contain iron or zinc. These paramagnetic rim lesions (PRL) are considered a promising pathological biomarker of iron/zinc accumulation in chronic active lesions and are identified using susceptibility-weighted imaging. They have altered morphology, sparse T- and B-cells at the core, and a slow rate of ongoing demyelination and axonal loss. Detection of PRL presently requires susceptibility-weighted imaging, as is known in the art. Haller et al., Susceptibility-weighted imaging: technical essentials and clinical neurologic applications Radiology 2021; 299:3-26.

Detection of slowly expanding/evolving lesions (SELs) on conventional T1-weighted/T2-weighted brain MRI provides an alternative readout of smoldering or chronic active plaques (Elliott et al. Slowly expanding/evolving lesions as a magnetic resonance imaging marker of chronic active multiple sclerosis lesions. Mult Scler J 2019; 25:1915-1925). Slowly expanding lesions have been described in the literature (Elliott et al. Chronic white matter lesion activity predicts clinical progression in primary progressive multiple sclerosis, Brain 2019; 142:2787-2799). SELs on conventional brain MRI are contiguous regions of T2 lesions showing constant and concentric local expansion as assessed by the Jacobian determinant of the non-linear deformation between the reference and follow-up scans. Generally, SELs are devoid of T1 gadolinium (Gd)-enhancement, have a lower mean T1 signal intensity at baseline and exhibit a progressive decrease in T1 intensity over time, compared to non-SEL areas of pre-existing lesions (Elliott et al. Ocrelizumab may reduce tissue damage in chronic active lesions as measured by change in T1 hypointensity of slowly evolving lesions in patients with primary progressive multiple sclerosis. Poster presented at AAN; Poster 376, Apr. 24, 2018; Los Angeles, CA).

As described and exemplified herein, the combination of these two radiological markers is informative of disease progression in earlier-stage MS patients, including patients known or suspected of having RIS, thereby enabling the identification of those patients likely to benefit from more aggressive disease-modifying antibody therapies, including anti-VLA-4 antibody therapies, earlier in the disease process, and consequent treatment initiation.

Moreover, these same earlier-stage patients may also benefit from the use of machine-learning based classifiers that can accurately and reproducibly discriminate acute from chronic MS lesions using unenhanced T1/T2 information from a single MRI scan, as described in Provisional Application Serial No. FR2103793, also filed on Apr. 13, 2021, and in co-pending International Application No. PCT/US2022/024450, the disclosures of which are expressly incorporated by reference herein. Accordingly, in some embodiments, the identification or one or more chronic active white matter lesions, and/or one or more SELs, in a single unenhanced MRI scan is also informative of disease progression in earlier-stage MS patients, including patients known or suspected of having RIS, thereby enabling the identification of those patients likely to benefit from more aggressive disease-modifying antibody therapies, including anti-VLA-4 antibody therapies, earlier in the disease process, and consequent treatment initiation.

### Definitions

A subject, as provided herein, is typically a male or female human subject (patient) who is undergoing or who will undergo treatment for a particular condition. The condition may be an autoimmune condition or an inflammatory condition. Often, autoimmune conditions are considered inflammatory conditions and vice versa, thus, in some embodiments the subject has an autoimmune condition and/or inflammatory condition. An autoimmune condition is a condition in which a subject's immune system attacks the subject's own cells/tissues. Non-limiting examples of the autoimmune conditions contemplated by the present invention include Radiologically Isolated Syndrome (RIS), and asymptomatic and/or early-stage multiple sclerosis (MS) (e.g., those having no diagnosed relapses).

Relapses in the context of MS occur in the absence of fever or infection and are not linked to environmental and systemic triggers; they denote acute inflammation in the CNS characterized by breach of integrity of the blood-brain barrier (BBB). In the radiological domain, the criteria for relapses are defined as an increase in lesion load/size on T2 imaging or T1 gadolinium enhancement of lesions on magnetic resonance imaging (MRI) in the brain, spinal cord or both. In the clinical domain, patients may present with “mild” symptoms such as e.g. pins and needles sensations that are fleeting and/or spasms that persist for a few seconds or minutes; alternatively or additionally, more severe exacerbations may include e.g. the occurrence of ataxia, visual deficits, diplopia, fatigue, cognitive impairment, bowel/bladder dysfunction, or motor weakness of a limb, which interfere with the patient's mobility, dexterity, ambulation, safety, or overall ability to function. The latter symptoms are more likely to result in a finding of relapse.

As used herein, “about” refers to within 0.1% to 5% of the given value (e.g., within 5%, 3%, 2%, 1%, 0.5%, 0.1% above or below the given value). Where amounts and other designated values are provided herein, the allowable deviation is within pharmaceutically acceptable standards.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein. A “pharmaceutically effective amount” or “therapeutically effective amount,” used interchangeably, is an amount sufficient to cure or at least partially arrest the symptoms of a disease and/or the complications of a disease.

A “disease-modifying antibody therapy” as contemplated herein for the treatment of CWMLA includes anti-VLA-4 antibodies, e.g., natalizumab and BIIB107, as well as anti-CD20 antibodies such as ocrelizumab. An “anti-VLA-4 antibody” is an anti-very late antigen (VLA)-4 monoclonal antibody, a humanized, a human, or a chimeric anti-VLA-4 monoclonal antibody. Anti-VLA-4 antibodies have been described in the art. They include, but are not limited to natalizumab and BIIB107, a monoclonal antibody that targets alpha-4 integrins and is currently under clinical investigation (ClinicalTrials.gov no. NCT04593121). See also PCT/US2011/032641 and PCT/US2019/034962, the disclosures of which are expressly incorporated by reference herein.

### Radiological Determinations

With the foregoing background in mind, in some embodiments the invention teaches the simultaneous or sequential acquisition of a combination of at least one T1- and T2-weighted image from a patient, for the identification of at least one SEL, and at least one susceptibility-weighted magnetic resonance image, for the identification of at least one PRL, and determining the extent of co-localization between the two, e.g. the percent of SEL that co-localize with PRLs, and vice-versa. In some embodiments, treatment with anti-VLA-4 therapy is initiated when at least 5%, 10%, 15%, 20%, 25%, 30%, 35%, 40%, 45%, 50%, 55%, 60%, 65%, 70%, or 75% of the patient's SELs co-localize with their PRLs. In some embodiments, treatment with anti-VLA-4 therapy is initiated when at least 5%, 10%, 15%, 20%, 25%, 30%, 35%, 40%, 45%, 50%, 55%, 60%, 65%, 70%, or 75% of the patient's PRLs co-localize with their SELs. Greater detail regarding specific embodiments of the invention is provided herein below.

In some embodiments, treatment with anti-VLA-4 therapy is initiated when at least 1%, 5%, 10%, 15%, 20%, 25%, 30%, 35%, 40%, 45%, 50%, 55%, 60%, 65%, 70%, or 75% of the patient's total T2 hyperintense lesion volume and/or number is identified as PRL. In some embodiments, treatment with anti-VLA-4 therapy is initiated when at least 1%, 5%, 10%, 15%, 20%, 25%, 30%, 35%, 40%, 45%, 50%, 55%, 60%, 65%, 70%, or 75% of the patient's total T2 hyperintense lesion volume and/or number is identified as SEL. In some embodiments, the SEL is detected using single time-point non-contrast T1- and T2-weighted MRI.

In various embodiments, the invention teaches a method for producing a series/set of brain images utilizing magnetic resonance imaging. In some embodiments, the method includes utilizing an MRI machine to apply a standardized 3-Tesla, 3D-isotropic multi-echo, gradient echo MRI to identify any PRLs in said patient, and a T1- and T2 weighted MRI to identify any SELs. The ‘co-localization’ between SELs and PRLs can be based on heuristic thresholds set from visual experience of the observer (radiologist) of the corresponding segmented volumes or alternatively rely on an automated processing pipeline determining the exact percent of SELs volume co-localizing with PRLs, and vice-versa.

One of skill in the art would readily appreciate that several different types of imaging systems could be used to perform the inventive methods described herein, including all of the types of imaging systems described in the examples and experiments set forth herein, as well as similar systems.

### Machine Learning Classification

In some embodiments the invention employs machine-learning based classifiers to classify MS lesions using unenhanced T1/T2 information from a single MRI scan, as described in Provisional Application Serial No. FR2103793 and co-pending International Application No. PCT/US2022/024450, the disclosures of which are expressly incorporated by reference herein for all purposes. Use of these classifiers may be able to effectively increase the sensitivity of single time-point acute MS lesion detection, and may be able to replicate, approach, or exceed the sensitivity of traditional detection of hyperintensities identified on a T1-weighted scan with gadolinium enhancement and/or of new hyperintense lesions on a T2-weighted scan in comparison with a prior reference scan, which may be reflective of new local inflammation.

In some embodiments, suitable methods of classifying brain lesions based on single point in time imaging can include: accessing patient image data from a single point in time; providing the patient image data as an input to a brain lesion classification model; generating a classification for each of one or more lesions identified in the patient image data; and providing the classification for each of the one or more lesions for display on one or more display devices; wherein the brain lesion classification model is trained using subject image data for a plurality of subjects, the subject image data being captured at two or more points in time. In some embodiments, the patient image data from the single point in time includes data from two or more image scan sequences. In some embodiments, the data from two or more image scan sequences include unenhanced MRI data, wherein the two or more image scan sequences do not include administration of paramagnetic contrast agents. In some embodiments, the classification for each of one or more lesions identified in the patient image data is selected to be one of acute or chronic, or SEL or non-SEL.

In some embodiments, certain radiomic features having predictive value with respect to the classification of a lesion as either acute or chronic and/or as SELs are utilized including e.g. the following exemplary embodiments:
Features that quantify the first order intensity of the core region of a lesion as it appears on a T2-weighted scan image. Such features account for acute lesions tending to be more intense than chronic lesions and more uniformly hyperintense, whereas chronic lesions may contain less hyperintense voxels.
Features that quantify the amount of signals appearing as low-gray around the periphery of a lesion as it appears on a T1-weighted scan image.
Features that quantify the amount of high-gray signals that are present in the periphery and/or core of a lesion as it appears on a T1-weighted scan image.
Features that relate to the inhomogeneity present in the images. For example, features may quantify the complexity of the image (the image is non-uniform and may include rapid changes in the gray levels), the variance of the gray levels with respect to a mean gray level, or the existence of homogenous patterns in the images.
Features that relate to the structure of the image, as relating to the presence of repeating patterns. For example, an image with more repeating patterns may be considered to be more “structured” than one with fewer observable intensity patterns.
Features that relate to the texture of the images, such as the coarseness or fineness of an image.

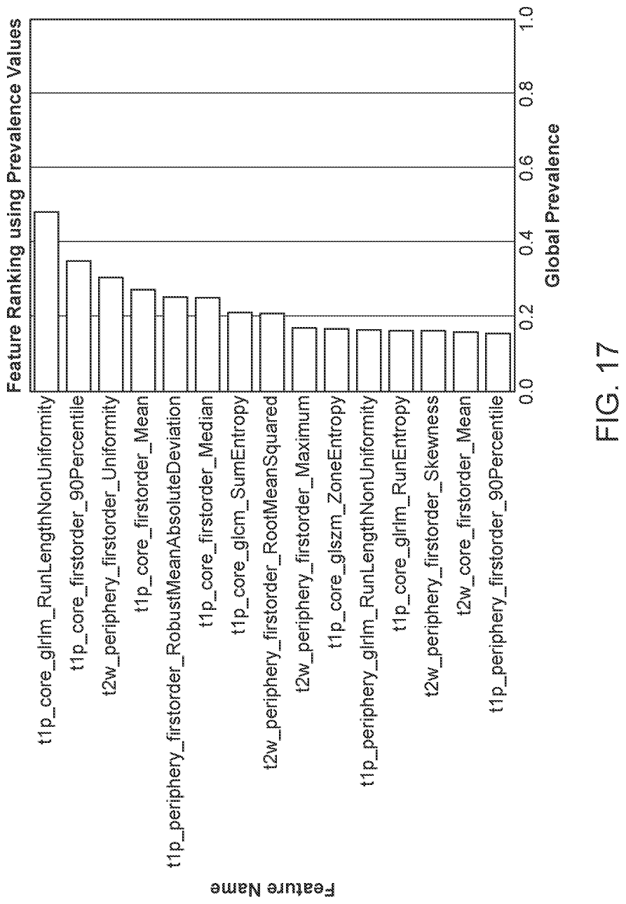

In some embodiments, the radiomic features for discriminating SEL from non-SEL are selected from the group comprising or consisting of the radiomic features listed in FIG. 17:
T1-weighted-MRI pre-contrast (extracted from non-contrast T1 weighted MRI image), computed within the core region, gray-level Run-Length-Matrix (quantifies gray level runs), run length non-uniformity (measures similarity of run lengths throughout the image, with a lower value indicating more homogeneity among run lengths in the image)
T1-weighted-MRI pre-contrast, computed within the core region, first order features (describe the distribution of voxel intensities within the image region defined by the mask through commonly used and basic metrics), 90th percentile of voxels in the patch
T2-weighted MRI (extracted from T2 weighted MRI image) computed within the periphery region, first order features, uniformity (a measure of the sum of the squares of each intensity value; a measure of homogeneity of the image array; greater uniformity implies a greater homogeneity or a smaller range of discrete intensity values)
T1-weighted-MRI pre-contrast, computed within the core region, first order features, mean (average gray level intensity within the patch)
T1-weighted-MRI pre-contrast, computed within the periphery region, first order features, robust mean absolute deviation (the mean distance of all intensity values from the Mean Value calculated on the subset of image array with gray levels in between, or equal to the 10th and 90th percentile)
T1-weighted-MRI pre-contrast, computed within the core region, first order features, median gray level intensity within the patch
T1-weighted-MRI pre-contrast, computed within the core region, gray-level Co-occurrence Matrix, sum entropy (sum of neighborhood intensity value differences)
T2-weighted MRI computed within the periphery region, first order values, root mean squared (the square-root of the mean of all the squared intensity values; another measure of the magnitude of the image values)
T2-weighted MRI computed within the periphery region, first order values, maximum gray level intensity within the patch
T1-weighted-MRI pre-contrast, computed within the core region, Gray-level Size Zone Matrix, zone entropy (measures the uncertainty/randomness in the distribution of zone sizes and gray levels; higher values indicate more heterogeneity in the texture patterns)
T1-weighted-MRI pre-contrast, computed within the periphery region, gray-level Run-Length-Matrix, run length nonuniformity (measures the similarity of run lengths throughout the image; a lower value indicates more homogeneity among run lengths in the image)
T1-weighted-MRI pre-contrast, computed within the core region, gray-level Run-Length-Matrix, run entropy (measures the uncertainty/randomness in the distribution of run lengths and gray levels; higher values indicate more heterogeneity in the texture patterns)
T2-weighted MRI, computed within the periphery region, first order values, skewness (measures asymmetry of distribution of values about the mean value; can be positive or negative)
T2-weighted MRI computed within the core region, first order values, mean (average gray level intensity within the patch)
T2-weighted MRI computed within the periphery region, first order values, 90th percentile of voxels in the patch

More generally, the machine learning classifier may employ one or more machine learning systems, methods, and/or models. A machine learning model may be considered as a model configured to receive input, and to apply one or more of a weight, bias, classification, or analysis on the input to generate an output. The output may include, for example, a classification of the input, an analysis based on the input, a design, process, prediction, or recommendation associated with the input, or any other suitable type of output. A machine learning model generally is trained using training data, e.g., experiential data and/or samples of input data, such as the types of training data described elsewhere herein, which are fed into the model in order to establish, tune, or modify one or more aspects of the model, e.g., the weights, biases, criteria for forming classifications or clusters, or the like. Aspects of a machine learning model may operate on an input linearly, in parallel, via a network (e.g., a neural network), or via any suitable configuration.

Training sets (e.g. subject image data captured at two or more points in time) may be used as inputs to train the machine learning classifier, may facilitate a selection or combination of machine learning methods, directed toward creating an optimal combination of such methods. One goal of such selection is the creation of an optimal subset of features to provide separation on a reduced imaging biomarker space between lesion types (e.g., acute versus chronic lesions or SEL versus non-SEL) or amount or degree of progression. In an embodiment, linear and non-linear feature-to-class correlation tests may be used to identify the features that account for the highest variance between the classifications.

This evaluation and classification may employ an initial feature ranking, such as shown in FIG. 17, and an initial feature selection that may, for example, identify a number of features with the strongest individual correlation. In an embodiment, there may be 50 such features selected as a feature subspace. From those features, embedded selection methods can leverage tree-based classifiers and sparse linear models including a least absolute shrinkage and selection operator (LASSO).

In this manner, starting from a feature subspace as just mentioned, it is possible to conduct a study, in the course of which a number of features in the feature subspace may be decremented by removing a feature, so as to eliminate the least useful feature at each step. In this fashion, an ensemble classifier can be optimized at each step. The process can proceed recursively, cycling between optimization and feature removal, to arrive at a ranking in which each decremented combination of radiomic features is associated with the lesion classification objective (e.g. active versus chronic or SEL versus non-SEL).

The outcome of this ensemble classification mechanism may be a selected subset of classification methods that may involve linear classification methods (e.g., logistic regression, support vector machines) and/or non-linear classification methods (e.g., perceptron, deep convolutional neural networks or other types of neural networks) which act to optimize separability between the two classes (active and chronic).

In an embodiment, a pool of machine learning models may undergo hyperparameter tuning via an extensive randomized grid search, which may be followed by a k-fold cross-validation on the classification task of interest. This tuning may then lead to a performance benchmark that can select the highest performing models, for example, the n top-performing models. These models may then be combined under a stacking or a winner takes all or a probabilistic importance sampling ensemble strategy.

Implementing a machine learning model may include deployment of one or more machine learning techniques, including various types of neural networks, and statistical techniques such as linear regression, logistical regression, random forest, or gradient boosted machine (GBM). Depending on the embodiment, training of the machine learning model may be supervised, or unsupervised, or both. Supervised learning may include providing training data and labels corresponding to the training data. Unsupervised training may include clustering, classification, or the like. Different types of clustering, or combinations of clustering, also may be used, and these may be supervised or unsupervised.

In an embodiment, a machine-learning based classifier may include one or more of a plurality of types of neural networks, including convolutional neural networks (CNN), deep or fully convolutional neural networks (DCNN, FCNN), deep learning neural networks (DNN), deep belief networks (DBN), and others with which ordinarily skilled artisans will be familiar.

A machine learning system which may be part of a machine learning model may include one or more processors, one or more storage devices intended for non-volatile non-transitory storage, and one or more memory devices, which may be volatile memory for transitory storage, but which also may include non-volatile memory for non-transitory storage. A plurality of machine learning methods, implemented by one or more machine learning systems, may be employed as part of the ensemble classification process. In an embodiment, the processors in a machine learning system may be graphics processing units (GPUs) or central processing units (CPUs), which can lend themselves to neural network structures or other learning frameworks.

Some literature distinguishes among machine learning, deep learning, artificial intelligence, and multiple instance learning in various ways. For purposes of the present discussion, any or all of these approaches may provide the necessary structure and functionality to accomplish one or more inventive goals as described herein.

### Dosing Regimens

The present disclosure also provides biphasic dosing regimens for reducing pathological inflammation with natalizumab, wherein the dosing regimens comprise an induction phase employing standard interval dosing (SID) followed by a chronic phase employing extended interval dosing (EID). In some embodiments, at least one treatment phase employs subcutaneous administration. In some embodiments, both treatment phases employ subcutaneous administration. In some embodiments, the same dose administered during the SID phase can be administered during the EID phase, and in some embodiments the same dose administered IV can be administered SC.

The biphasic dosing regimen contemplated herein refers to the administration of natalizumab in at least two phases, e.g., an induction phase and a chronic phase. In some embodiments, the induction phase comprises administration of natalizumab on an SID schedule and the chronic phase comprises administration of natalizumab on an EID schedule. In some embodiments, the induction phase comprises administration of natalizumab once every 2 weeks, about once every 2 weeks, once every 3 weeks, about once every 3 weeks, once every 4 weeks, about once every 4 weeks, once every 30 days, about once every 30 days, once a month or about once a month for at least 6 months, for at least 8 months, for at least 10 months, or for at least 12 months. In some embodiments, the induction phase is from 6 to 18 months, from 8 to 16 months, from 10 to 14 months, is 11 months, is 12 months, or is 13 months. In some embodiments, the chronic phase comprises administration of natalizumab once every 5 to 10 weeks. In some embodiments, the chronic phase comprises administration of natalizumab every 5 weeks, about every 5 weeks, every 6 weeks, about every 6 weeks, every 7 weeks, about every 7 weeks, every 8 weeks, or about every 8 weeks. In some embodiments, both the induction phase and the chronic phase comprise SC administration. In some embodiments, the induction phase and the chronic phase are solely SC administration. In some embodiments, surprisingly, the SC dosing and amount of natalizumab can be consistent with IV dosing.

In some embodiments, the therapeutically effective amount administered during the induction phase and the chronic phase are the same, and the therapeutically effective amount is between 250-450 mg (e.g., 250 mg, 300 mg, 350 mg, 400 mg, or 450 mg), more preferably about 300 mg, still more preferably 300 mg. In some embodiments, the therapeutically effective amount administered SC during the chronic phase is between 300-500 mg (e.g., 300 mg, 350 mg, 400 mg, 450 mg, or 500 mg). In some embodiments, the therapeutically effective amount is between about 250-about 450 mg (e.g., about 250 mg, about 300 mg, about 350 mg, about 400 mg, or about 450 mg), more preferably about 300 mg, still more preferably 300 mg. In some embodiments, the therapeutically effective amount administered SC during the chronic phase is between about 300-about 500 mg (e.g., about 300 mg, about 350 mg, about 400 mg, about 450 mg, or about 500 mg), more preferably about 300 mg, still more preferably 300 mg. Representative biphasic dosing regimens are disclosed in U.S. Provisional Application Ser. No. 63/113,864 (filed Nov. 14, 2020), 63/113,865 (filed Nov. 14, 2020), 63/142,968 (filed Jan. 28, 2021), 63/142,970 (filed Jan. 28, 2021), and co-pending International Application No. PCT/US2021/059266, the disclosures of which are expressly incorporated by reference herein.

The present disclosure also provides a chronic dosing regimen for reducing pathological inflammation with natalizumab. In some embodiments, natalizumab is administered in a chronic dose regimen, wherein the chronic dosing regimen comprises administration of natalizumab at a fixed interval of every 4 weeks. In some embodiments, the chronic dosing regimen is a fixed, non-weight based amount of natalizumab. In some embodiments, a therapeutically effective amount of natalizumab is between about 250-450 mg, more preferably about 300 mg, still more preferably a fixed, non-weight based dose of 300 mg. In further embodiments, the chronic dosing regimen is every 4 weeks for a period of about 4 months, about 5 months, about 6 months, about 7 months, about 8 months, about 9 months, about 10 months, about 11 months, about 12 months, or longer than about 12 months. Representative chronic dosing regimens are disclosed in WO 2003/072040, the disclosure of which is expressly incorporated by reference herein.

In some embodiments, the chronic dosing regimen comprises SC injection. In some embodiments, the chronic dosing regimen comprises IV administration. Representative SC administration formulations are disclosed in WO 2008/157356, the disclosure of which is expressly incorporated by reference herein.

Relevant biomarkers for determining and/or monitoring efficacy of the treatment protocols provided herein include, e.g., sVCAM and/or Nf-L. Without being bound by theory, increased saturation and/or occupancy by natalizumab of its target α4 integrin on the surface of circulating lymphocytes leads to decreased surface expression of α4-integrin on lymphocytes, as well as decreased serum concentration of sVCAM. Correspondingly, sVCAM provides an effective surrogate biomarker for α4-integrin receptor saturation, and for immune surveillance activity in general, see, e.g. Plavina et al., Neurology (2017) 89(15):1584-1593. Neurofilament proteins such as Nf-L, in contrast, provide an indication of axonal damage and neuronal death, and serve as effective surrogate biomarkers for ongoing disease activity in MS patients in particular. See, e.g., Kuhle et al. Mult Scler. (2013) 19:1597-603; Varhaug et al., Front Neurol. (2019) 10: 338.

These biphasic dosing regimens are provided for increasing the safety of natalizumab therapy. In some embodiments, the biphasic dosing regimens are provided for increasing the safety of chronic natalizumab therapy. Safety may be increased by reducing the risk of an adverse event, e.g. PML. In some cases, the biphasic regimen reduces the risk of PML, reduces the risk of inducing generation of anti-natalizumab antibodies, reduces the risk of patient sensitization to natalizumab, or a combination thereof. In some cases, the biphasic regimen reduces the risk of loss of efficacy of natalizumab treatment due to the generation of anti-idiotypic antibodies to natalizumab in the patient.

Patients who are seropositive for anti-JCV antibodies are at a particularly high risk of PML. In some embodiments, a PML risk subject has an anti-JCV antibody index level (e.g., a mean index level) of greater than 1.5. In some embodiments, a low PML risk subject is a subject who has an anti-JCV antibody index level (e.g., a mean index level) of less than or equal to 0.9. Anti-JC virus index values are calculated from a two-step ELISA antibody assay of serum/plasma (STRATIFY JCV™ Antibody (with Index) with Reflex to Inhibition Assay; see, e.g., Lee, P. et al. J of Clin Virol, 2013; 57(2): 141-146, incorporated herein by reference). Antibody index level, assays for assessing index level, and the use of such index levels and assays, for determining PML risk are described in, e.g., WO 2012/166971 and WO 2014/193804.

A subject may be considered a high PML risk if the subject tested seropositive for anti-JCV antibodies prior to commencement of natalizumab therapy, or if the subject switches from a seronegative anti-JCV antibody status to a seropositive anti-JCV antibody status during natalizumab therapy. In some embodiments, a subject is considered a high PML risk if the subject has an anti-JCV antibody index level of greater than 1.5 prior to commencement of natalizumab therapy, or if the subject switches from a lower anti-JCV antibody index level of less than or equal to 0.9 to a higher anti-JCV antibody index level of greater than 1.5 during natalizumab therapy. For example, prior to starting natalizumab therapy, a subject may be tested for the presence or absence of anti-JCV antibodies. If the test results indicate that the subject is a low PML risk subject (seronegative for anti-JCV antibodies, or having an anti-JCV antibody index level of less than or equal to 0.9), then the subject may be identified as a subject for natalizumab therapy on a SID schedule of 4-week intervals. During the course of the natalizumab therapy on a SID schedule, the subject may be re-tested for the presence or absence of anti-JCV antibodies (e.g., tested every month or every 2, 3, 4, 5 or 6 months, or every year). If upon re-testing the subject has switched from seronegative to seropositive for anti-JCV antibodies, or from having an anti-JCV antibody index level of less than or equal to 0.9 to having an anti-JCV antibody index level of greater than 1.5, then the subject may be identified as a subject for natalizumab therapy on an EID schedule of at least 5-week intervals.

### EXAMPLES

### Example 1: Association Between Chronic White Matter Lesion Activity and Disability Progression in SPMS Patients with or without Acute Inflammation

Objective: Slowly expanding lesions (SELs), a subgroup of white matter lesions that gradually expand over time, have been shown to predict disability accumulation in primary progressive multiple sclerosis (MS) disease. The relationships between SELs, acute white matter lesion activity (AWMLA), chronic white matter lesion activity (CWMLA), and disability progression are not well understood. This study assessed CWMLA and acute lesion activity (AWMLA) in the brain white matter of a secondary progressive MS population.

CWMLA was measured in this study by the change in T1LV from baseline to week 108 in SELs, non-SELs, and total pre-existing chronic nonenhancing T2 (CNT2) lesions. AWMLA was defined by having either 1) gadolinium-enhancing (Gd+) T1 lesions at any time point in the trial up to week 108, including baseline, or 2) any postbaseline new or enlarging T2 lesions. The following was examined in a secondary progressive MS population treated with placebo: 1) the association between T1-weighted (T1w) MRI features of CWMLA in SELs and non-SELs and confirmed disability progression, 2) the association between CWMLA and confirmed disability progression in the absence of AWMLA, and 3) the association between CWMLA and AWMLA. In this study, we also examined the ASCEND phase 3 clinical trial (ClinicalTrials.gov no. NCT01416181), which compared natalizumab with placebo in secondary progressive MS (SPMS).

### Materials and Methods

### Trial Design, Patients, and MRI

The ASCEND study (ClinicalTrials.gov no. NCT01416181) was a two-part, multicenter, randomized, double-blind, placebo-controlled phase 3 study in patients with SPMS to assess the efficacy and safety of natalizumab. Details of the study design and outcomes have previously been described in detail. Kapoor et al. Lancet Neurol 2018; 17:405-415. Axial T1W (3D Spoiled gradient echo: TR=28-35 ms; TE=4-11 ms; flip angle=27°-30°; resolution 0.98×0.98×3 mm) and Axial T2W (2D Fast Spin Echo: TR=4000-7400 ms; TE=58-95 ms; resolution=0.98×0.98×3 mm) were acquired at baseline, week 24, week 48, week 72, week 96, and week 108. The SEL analysis population represents the subset of the intention-to-treat population that had available T1w and T2-weighted (T2w) images at all time points from baseline to week 108 (including weeks 24, 48, 72, and 96).

### Clinical Measures of Disability Progression

Expanded Disability Status Scale (EDSS), Timed 25-Foot Walk (T25FW), and 9-Hole Peg Test (9HPT) assessments were performed at baseline and every 12 weeks through week 108. Composite confirmed disability progression was defined as meeting one or more of the following three criteria: an increase of ≥1.0 point from a baseline EDSS score ≤5.5 or an increase of ≥0.5 point from a baseline score ≥6.0, an increase of ≥20% from baseline in T25FW time, and/or an increase of ≥20% from baseline in 9HPT time (on either hand). Progression was confirmed at a subsequent visit ≥6 months after the possible start of progression and at the end of the trial. To minimize the possibility of capturing disability progression due to clinical relapses, included confirmed disability progression events could not have started or been confirmed ≤74 days after onset of an independent neurology evaluation committee-confirmed clinical relapse. Absence of acute lesion activity was defined as no baseline or postbaseline T1 gadolinium-enhancing (Gd+) lesions and no postbaseline new or enlarging T2-hyperintense lesions.

### Identification of SELs, AWMLA, and Overall CWMLA

The process of SEL identification has been described in detail elsewhere. Elliott et al. Brain 2019; 142:2787-2799 and Elliott et al. Mult Scler 2019; 25:1915-1925. Briefly, SELs are contiguous regions of preexisting T2 lesions showing constant and concentric local expansion from baseline to week 108. Prior to SEL detection, T2 lesions were identified in baseline scans using a semi-automated method in which a fully automated segmentation of T2 lesions was subsequently manually reviewed and corrected by a single trained MRI reader. Francis S J. In: McGill University DoN, ed., 2005. In the first stage of SEL detection, SEL candidates are identified as contiguous regions of ≥10 voxels in the baseline T2 lesion mask that a) are not Gd+ and b) show a minimum local volumetric expansion, as determined by the Jacobian determinant of the nonlinear deformation between the baseline and week 108 scans. The second stage of SEL detection scores each SEL candidate in turn on the basis of the concentricity and constancy of expansion across time. Considering local expansion between baseline and each intermediate time point (weeks 24, 48, 72, and 96) allows for the identification of SEL candidates undergoing constant and gradual expansion across time, while measuring concentricity allows for the identification of SEL candidates exhibiting inside-out radial expansion. Each SEL candidate is assigned a SEL score, calculated as the sum of the mean normalized measures for constancy and concentricity.

SELs were identified in SPMS patients from the ASCEND phase 3 clinical trial (ClinicalTrials.gov no. NCT01416181). Non-SELs, defined as the portion of the nonenhancing baseline T2 lesion mask not identified as SELs, were also assessed, as were the totality of nonenhancing T2 lesions. The ASCEND SEL analysis population (placebo, n=292; natalizumab, n=308) represents the subset of patients who had available T1w and T2w images at all time points from baseline to week 108. Results are presented for SELs with SEL score ≥0. The distribution of SELs and non-SELs by sex, baseline EDSS, age, and disease duration are provided in Tables 1-3.

TABLE 1
SEL Count

| Baseline Age, Years | <=45 | >45-52 | >52 |
|---|---|---|---|
| n | 210 | 206 | 184 |
| Mean | 7.5429 | 8.0825 | 7.5435 |
| Standard deviation | 8.83991 | 11.39396 | 10.69489 |
| Median | 4.0000 | 4.0000 | 3.5000 |
| 25%, 75% percentile | 1.0000, | 1.0000, | 1.0000, |
| | 12.0000 | 10.0000 | 11.0000 |
| Min, Max | 0.000, | 0.000, | 0.000, |
| | 42.000 | 78.000 | 61.000 |

| Years since First MS symptoms | <=8 | >8-15 | >15 |
|---|---|---|---|
| n | 206 | 213 | 177 |
| Mean | 7.0437 | 8.0282 | 8.113 |
| Standard deviation | 9.28324 | 10.38364 | 11.37675 |
| Median | 3.0000 | 4.0000 | 4.0000 |
| 25%, 75% percentile | 1.0000, | 1.0000, | 1.0000, |
| | 9.0000 | 12.0000 | 9.0000 |
| Min, Max | 0.000, | 0.000, | 0.000, |
| | 42.000 | 61.000 | 78.000 |

| Baseline EDSS | High EDSS ≥ 6.0 | Low EDSS <6.0 |
|---|---|---|
| n | 361 | 239 |
| Mean | 8.5014 | 6.5607 |
| Standard deviation | 11.05972 | 9.01466 |
| Median | 4.0000 | 3.0000 |
| 25%, 75% percentile | 1.0000, 12.0000 | 1.0000, 8.0000 |
| Min, Max | 0.000, 61.000 | 0.000, 78.000 |

| Gender | Female | Male |
|---|---|---|
| n | 381 | 219 |
| Mean | 8.2572 | 6.8082 |
| Standard deviation | 10.65324 | 9.69677 |
| Median | 4.0000 | 3.0000 |
| 25%, 75% percentile | 1.0000, 11.0000 | 1.0000, 8.0000 |
| Min, Max | 0.000, 78.000 | 0.000, 59.000 |

TABLE 2
SEL Volume, T1-weighted

| Baseline Age, Years | <=45 | >45-52 | >52 |
|---|---|---|---|
| n | 210 | 206 | 184 |
| Mean, mm³ | 688.81 | 748.97 | 799.79 |
| Standard deviation, mm³ | 1472.156 | 1552.809 | 2127.059 |
| Median, mm³ | 117.1 | 200.29 | 111.44 |
| 25%, 75% percentile, mm³ | 5.72, | 5.72, | 2.86, |
| | 738.20 | 586.55 | 708.16 |
| Min, Max, mm³ | 0.0, | 0.0, | 0.0, |
| | 14131.7 | 10809.8 | 23708.3 |
| Years since First MS | | | |
| symptoms | <=8 | >8-15 | >15 |
| n | 206 | 213 | 177 |
| Mean, mm³ | 750.11 | 680.26 | 812.86 |
| Standard deviation, mm³ | 1748.405 | 1178.017 | 2198.383 |
| Median, mm³ | 101.00 | 145.92 | 145.92 |
| 25%, 75% percentile, mm³ | 0.00, | 5.72, | 31.47, |
| | 595.14 | 726.76 | 515.02 |
| Min, Max, mm³ | 0.0, | 0.0, | 0.0, |
| | 14131.7 | 8123.1 | 23708.3 |

| Baseline EDSS | High EDSS ≥ 6.0 | Low EDSS <6.0 |
|---|---|---|
| n | 361 | 239 |
| Mean, mm³ | 848.52 | 584.86 |
| Standard deviation, mm³ | 1944.682 | 1304.472 |
| Median, mm³ | 151.65 | 105.87 |
| 25%, 75% percentile, mm³ | 8.58, 738.20 | 2.86, 517.88 |
| Min, Max, mm³ | 0.0, 23708.3 | 0.0, 14131.7 |

| Gender | Female | Male |
|---|---|---|
| n | 381 | 219 |
| Mean, mm³ | 791.37 | 660.22 |
| Standard deviation, mm³ | 1631.066 | 1870.948 |
| Median, mm³ | 151.65 | 103.00 |
| 25%, 75% percentile, mm³ | 14.31, 663.81 | 0.00, 595.14 |
| Min, Max, mm³ | 0.0, 14131.7 | 0.0, 23708.3 |

TABLE 3
non-SEL Volume, T1-weighted

| Baseline Age, Years | <=45 | >45-52 | >52 |
|---|---|---|---|
| n | 210 | 206 | 184 |
| Mean, mm³ | 4703.94 | 4785.52 | 4949.89 |
| Standard deviation, mm³ | 5107.976 | 5174.203 | 6318.338 |
| Median, mm³ | 2658.10 | 3107.31 | 2778.27 |
| 25%, 75% percentile, mm³ | 1084.41, | 1178.83, | 816.89, |
| | 7027.21 | 6878.43 | 5945.66 |
| Min, Max, mm³ | 34.3, | 8.6, | 0.0, |
| | 27333.5 | 31751.2 | 45313.5 |
| Years since First | | | |
| MS symptoms | <=8 | >8-15 | >15 |
| n | 206 | 213 | 177 |
| Mean, mm³ | 4184.01 | 4811.28 | 5563.19 |
| Standard deviation, mm³ | 5141.369 | 5246.273 | 6203.788 |
| Median, mm³ | 2123.04 | 2969.97 | 3525.05 |
| 25%, 75% percentile, mm³ | 603.72, | 1010.02, | 1599.43, |
| | 5808.32 | 6712.48 | 7264.70 |
| Min, Max, mm³ | 8.6, | 0.0, | 68.7, |
| | 27851.3 | 31751.2 | 45313.5 |

| Baseline EDSS | High EDSS ≥ 6.0 | Low EDSS <6.0 |
|---|---|---|
| n | 361 | 239 |
| Mean, mm³ | 4938.26 | 4609.67 |
| Standard deviation, mm³ | 5491.272 | 5569.331 |
| Median, mm³ | 3061.53 | 2509.31 |
| 25%, 75% percentile, mm³ | 1078.69, 6878.43 | 875.54, 6434.93 |
| Min, Max, mm³ | 0.0, 45313.5 | 8.6, 30189.0 |
| Gender | Female | Male |
| n | 381 | 219 |
| Mean, mm³ | 5049.94 | 4385.38 |
| Standard deviation, mm³ | 5860.391 | 4856.158 |
| Median, mm³ | 3061.53 | 2615.18 |
| 25%, 75% percentile, mm³ | 841.21, 7138.80 | 1101.58, 5851.24 |
| Min, Max, mm³ | 22.9, 45313.5 | 0.0, 27851.3 |

AWMLA was defined by having either 1) Gd+ T1 lesions at any time point in the trial up to week 108, including baseline, or 2) any postbaseline new or enlarging T2 lesions. Gd+ T1 lesions were determined as a consensus of 2 fully manual identifications by 2 trained MRI readers, where any discrepancies were adjudicated by a third independent reader. New or enlarging T2 lesions were determined by comparing T2 lesion masks at successive timepoints and automatically identifying focal areas of new T2 lesions, which were not present at the previous timepoint and showed a minimum increase in T2-weighted intensity. These focal areas of new T2 lesions could be entirely in NAWM (new) or adjacent to pre-existing T2 lesions (enlarging). All automatically identified new or enlarging T2 lesions were manually reviewed and corrected where necessary.

It is important to appreciate that the SEL approach to the detection of lesion expansion is fundamentally different from that used for the detection of so-called new ‘T2 enlarging lesions’, commonly reported in counts of ‘new or enlarging T2 lesions’ in clinical trials. Arnold et al. Mult Scler 2021; 27(11): 1681-3. Methods for the detection of ‘enlarging’ lesions in the context of ‘new or enlarging T2 lesion counts’ (which vary from laboratory to laboratory) have been designed to detect what are essentially new foci of acute white matter lesion activity that are connected by adjacency to areas of pre-existing T2-signal abnormality and therefore may not qualify as ‘de novo’ new lesions (which by definition have to be surrounded by normal-appearing WM). CWMLA was measured by the change in T1-hypointense lesion volume (T1LV) from baseline to week 108 in SELs, non-SELs, and total pre-existing chronic nonenhancing T2 (CNT2) lesions in SPMS patients in the placebo arm. T1-hypointense lesions were defined as areas of T2-lesion not showing gadolinium enhancement and with T1-weighted intensity less than or equal to median T1-weighted intensity of gray matter.

Whole brain atrophy was measured via Jacobian integration. Nakamura et al. Neuroimage Clin 2014; 4: 10-17.

### Statistical Analyses

The statistical analysis of SEL data was exploratory and included all patients from ASCEND with no missing or nonevaluable T1w and T2w scans at any time point (baseline to week 108; SEL analysis population). No imputation of missing data was performed.

A two-sample proportion test was applied to compare baseline Gd+ T1 lesions between the two treatment groups. CWMLA was compared between progressors and nonprogressors using the Van Elteren test, stratified for progression status, baseline EDSS score (≤5.5 or ≥6.0), and baseline T2-hyperintense lesion volume (T2LV) category based on tertiles (≤6908.79 mm³, >6908.79-18818.49 mm³, and >18,818.49 mm³). Analyses of the association between AWMLA and SEL prevalence were based on the Van Elteren test, stratified for AWMLA, baseline EDSS score (≤5.5 or ≥6.0), and baseline T2LV category based on tertiles (≤6908.79 mm³, >6908.79-18818.49 mm³, and >18818.49 mm³). Comparisons of CWMLA between placebo and natalizumab were based on the Van Elteren test, stratified for treatment, baseline EDSS score (≤5.5 or ≥6.0), and baseline T2LV category based on tertiles (≤6908.79 mm³, >6908.79-18818.49 mm³, and >18818.49 mm³). Statistical tests were two-sided and conducted at the 5% significance level without adjustment for multiplicity.

### Results

### Baseline Demographics and Brain MRI Characteristics of the SPMS Analysis Population

The baseline demographics and brain MRI characteristics of the analysis population available for SEL detection and the intention-to-treat population from the ASCEND study dataset are presented in Table 4. Age and gender were distributed similarly across the SEL analysis and intention-to-treat populations and between treatment arms. In the SEL analysis population, a greater percentage of natalizumab—than placebo-treated individuals had ≥1 Gd+ T1 lesion at baseline (28% vs 19%), but the difference was not statistically significant. In the SEL analysis population, the natalizumab- and placebo-treated groups had a similar mean T2LV at baseline (18.1 vs 16.5 cm³). Mean normalized brain volume at baseline was also similar in the two treatment groups.

TABLE 4
Baseline Characteristics of ASCEND Analysis Population

| | SEL Analysis Population | | ITT Population | |
|---|---|---|---|---|
| Baseline | Placebo | Natalizumab | Placebo | Natalizumab |
| Characteristics | (n = 292) | (n = 308) | (n = 448) | (n = 439) |
| Age, mean (SD) | 47.8 (7.6) | 47.4 (7.2) | 47.2 (7.8) | 47.3 (7.4) |
| Female, % | 65 | 62 | 63 | 62 |
| Patients with ≥1 | 19 | 28 | 22 | 26 |
| Gd+ T1 lesion, % | | | | |
| Mean (SD) T2LV, | 16.5 | 18.1 (18.5) | 16.2 | 17.4 (17.6) |
| cm³ | (16.7) | | (16.4) | |
| Normalized brain | 1429.8 | 1422.0 | 1425.8 | 1420.9 |
| volume, mean | (81.64) | (81.75) | (83.1) | (82.8) |
| (SD), cm³ | | | | |

ASCEND SEL analysis population represents the subset of the ITT population that had available T1- and T2-weighted images at all time points from baseline to week 108 (including weeks 24, 48, 72, and 96).
Gd+—gadolinium enhancing; ITT—intention to treat; SD—standard deviation; SEL—slowly expanding lesion; T2LV—T2-hyperintense lesion volume.

Disability Progression in SPMS was Associated with Greater CWMLA in the Placebo Arm

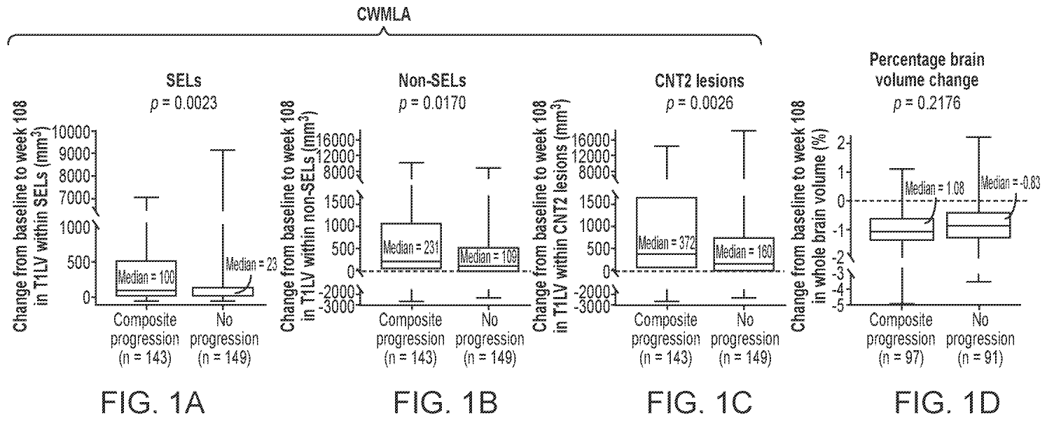

CWMLA was measured by the change in T1LV from baseline to week 108 in SELs, non-SELs, and total preexisting CNT2 lesions in SPMS patients treated with placebo. The analysis was restricted to the placebo group to avoid treatment effects, as natalizumab decreases AWMLA. CWMLA was compared in patients with composite disability progression (n=143) and patients who remained progression free (n=149). SPMS patients with confirmed composite disability progression had significantly more severe CWMLA than those who were progression free, as measured by T1LV change within SELs, non-SELs, and CNT2 lesions (median increase [Q1, Q3] progressors vs non-progressors: 100 [3, 524] vs 23 [0, 155] mm³, p=0.0023; 231 [17, 1090] vs 109 [−29, 538] mm³, p=0.0170; and 372 [26, 1662] vs 160 [−23, 770] mm³, p=0.0026, respectively; FIG. 1A-C). In contrast, the brain atrophy rate as measured by whole brain volume change from baseline to week 108 did not differ significantly between SPMS patients with and without composite confirmed disability progression (p=0.2176; FIG. 1D).

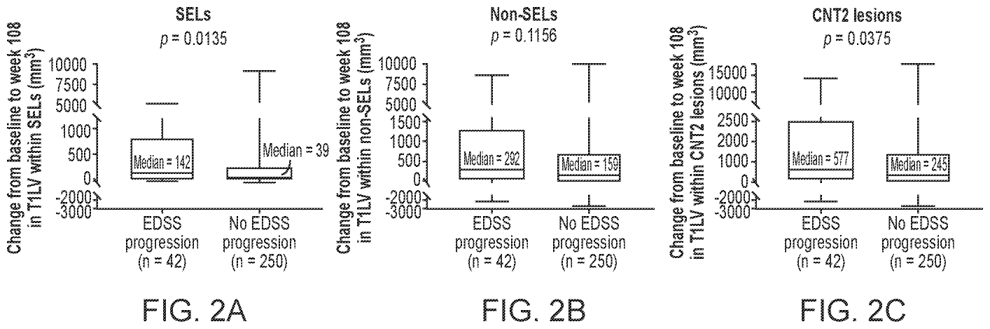

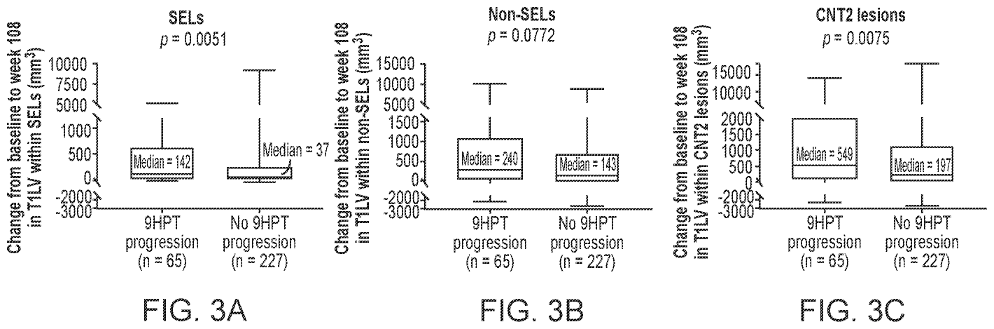

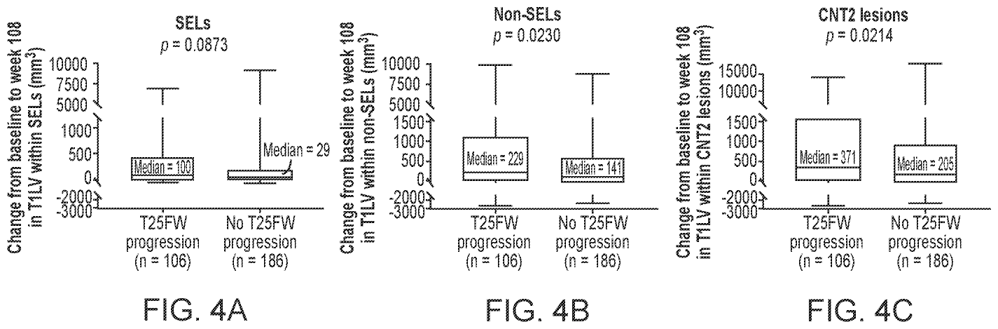

When confirmed disability progression was based only on EDSS score, CWMLA as measured by T1LV increase was significantly greater in progressors than nonprogressors within SELs (median increase [Q1, Q3] progressors vs non-progressors: 142 [6, 815] vs 39 [0, 258] mm³, p=0.0135; FIG. 2A) and CNT2 lesions (median increase [Q1, Q3] progressors vs non-progressors: 577 [66, 2529] vs 246 [0, 1090] mm³, p=0.0375; FIG. 2C), with a consistent trend in the same direction in non-SELs (median increase [Q1, Q3] progressors vs non-progressors: 292 [26, 1302] vs 159 [−9, 710] mm³, p=0.1156; FIG. 2B). Similarly, when confirmed disability progression was based only on 9HPT score, CWMLA as measured by T1LV increase was significantly greater in progressors than nonprogressors within SELs (median increase [Q1, Q3] progressors vs non-progressors: 112 [0, 629] vs 37 [0, 258] mm³, p=0.0051; FIG. 3A) and CNT2 lesions (median increase [Q1, Q3] progressors vs non-progressors: 549 [66, 1995] vs 197 [0, 1139] mm³, p=0.0075; FIG. 3C) and numerically greater in progressors than nonprogressors within non-SELs (median increase [Q1, Q3] progressors vs non-progressors: 240 [−9, 1097] vs 143 [−3, 661] mm³, p=0.0772; FIG. 3B). Finally, when confirmed disability progression was based only on T25FW, CWMLA as measured by T1LV increase was significantly greater in progressors than nonprogressors within non-SELs (median increase [Q1, Q3] progressors vs non-progressors: 229 [20, 1133] vs 141 [−25, 609] mm³, p=0.0230; FIG. 4B) and CNT2 lesions (median increase [Q1, Q3] progressors vs non-progressors: 371 [23, 1634] vs 205 [−6, 954] mm³, p=0.0214; FIG. 4C) and numerically greater in progressors than nonprogressors within SELs (median increase [Q1, Q3] progressors vs non-progressors: 100 [3, 426] vs 29 [0, 220] mm³, p=0.0873; FIG. 4A).

Disability Progression in SPMS Remained Associated with Greater CWMLA in the Complete Absence of AWMLA in the Placebo Arm

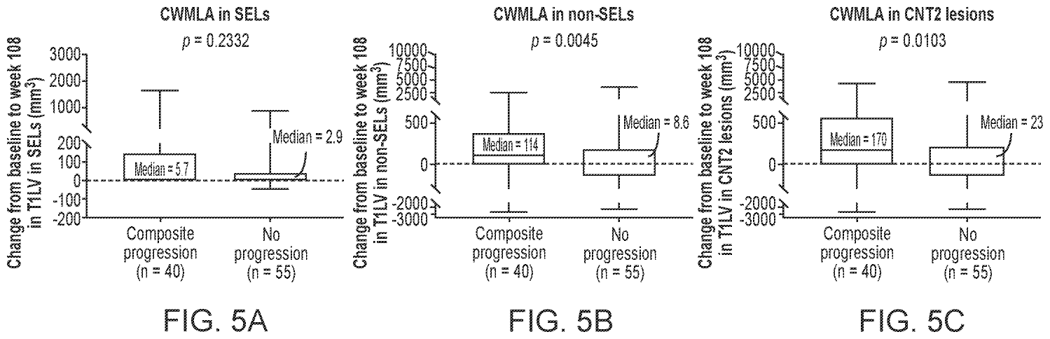

From baseline to week 108, 95 of 292 placebo SPMS patients had no AWMLA, defined as no baseline or postbaseline Gd+ T1 lesions and no postbaseline new/enlarging T2 lesions. CWMLA in SPMS patients with no AWMLA was compared in patients with composite confirmed disability progression (n=40) and patients who remained progression free (n=55). Patients exhibiting composite progression had more severe CWMLA than those who were progression free, as shown by a significant difference in T1LV change within non-SELs (p=0.0045; FIG. 5B) and CNT2 lesions (p=0.0103; FIG. 5C), and a trend in the same direction was observed in SELs (p=0.2332; FIG. 5A).

SEL Prevalence in SPMS Patients Treated with Placebo was Lower in the Absence of AWMLA

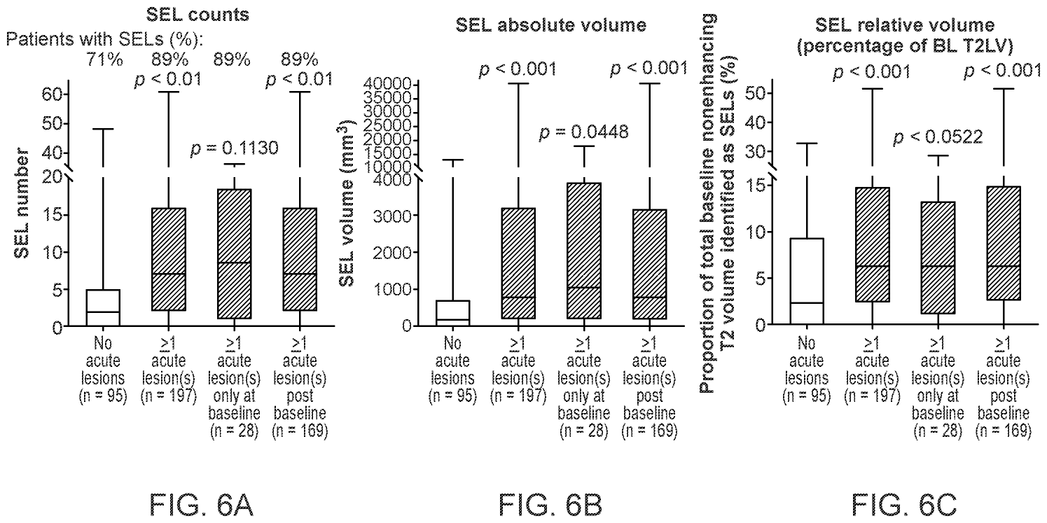

Placebo SPMS patients with AWMLA had a higher SEL prevalence as measured by SEL number and volume (based on T2w borders of SELs at baseline) and a higher proportion of preexisting baseline T2LV identified as SELs than patients with no AWMLA (FIG. 6A-C). The proportion of patients with at least one SEL was also higher in patients with AWMLA (89%, n=197) compared to those with no AWMLA (71%, n=95). Analysis of the differences in SEL prevalence between SPMS patients with no AWMLA (n=95) and those with AWMLA at baseline only (n=28) or post baseline (n=169) confirmed that both baseline and postbaseline AWMLA were associated with a higher SEL prevalence, though the sample size of patients with AWMLA at baseline only was too small for the associated SEL prevalence to reach significance with respect to SEL number and relative volume (FIG. 6A and C). An analysis of the frequency distribution of patients by range of SEL prevalence also showed that 19% of patients with AWMLA had >20% of their total T2 lesion burden identified as SELs, compared with only 5% of patients with no AWMLA (FIG. 6D).

### Natalizumab Versus Placebo Effect on SELs and CWMLA in SPMS Patients

The placebo- and natalizumab-treated arms had similar percentages of patients with ≥1 SEL detected from baseline to week 108 (83% vs 79%).

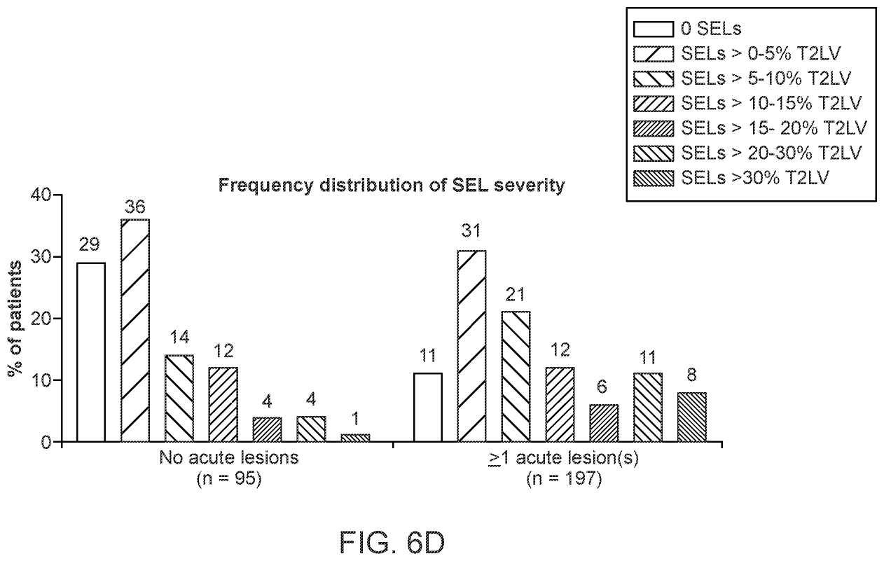

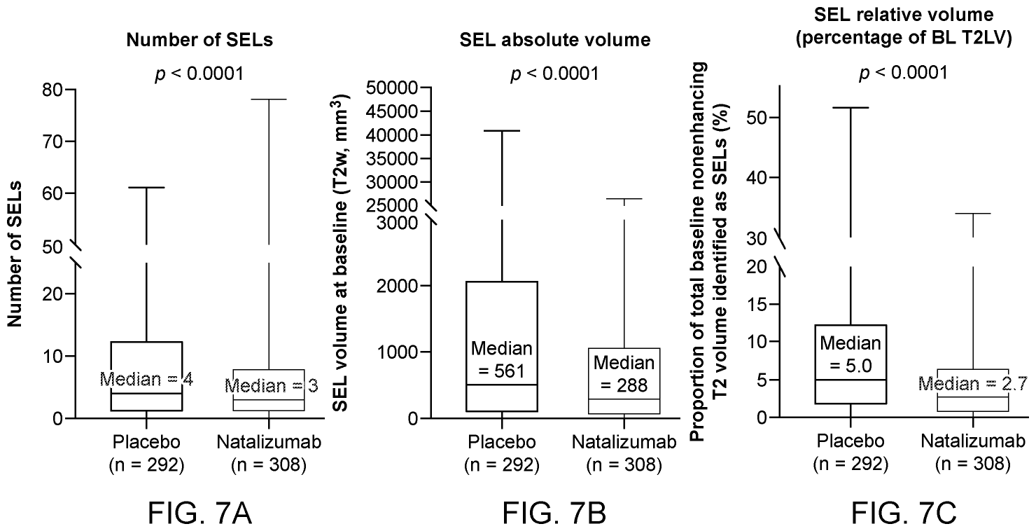

Natalizumab was associated with lower SEL prevalence than placebo, as indicated by a lower number of SELs (median number, 3 vs 4; p<0.0001; FIG. 7A) and a lower absolute SEL volume (median T2w SEL volume at baseline, 288 vs 561 mm³; p<0.0001; FIG. 7B). Accordingly, the proportion of total baseline nonenhancing T2LV longitudinally identified as SELs was significantly lower in natalizumab—than placebo-treated patients (median proportion, 2.7% vs 5.0%; p<0.0001; FIG. 7C).

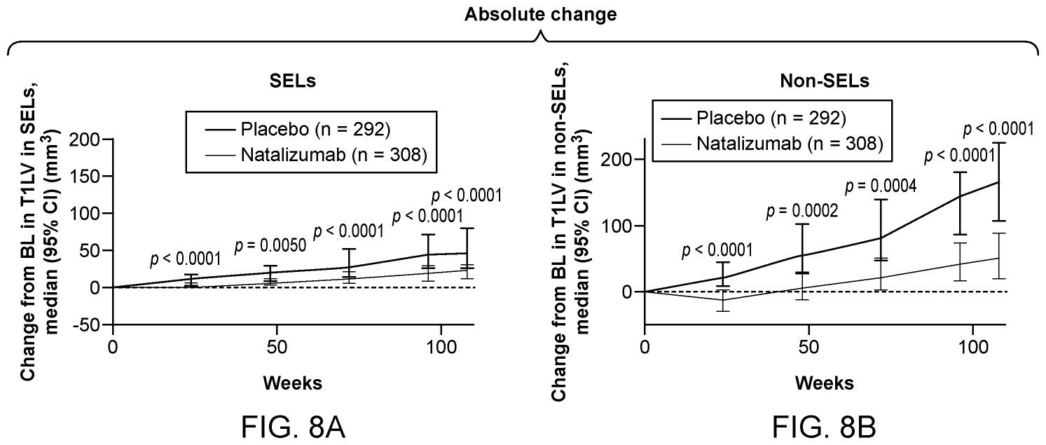

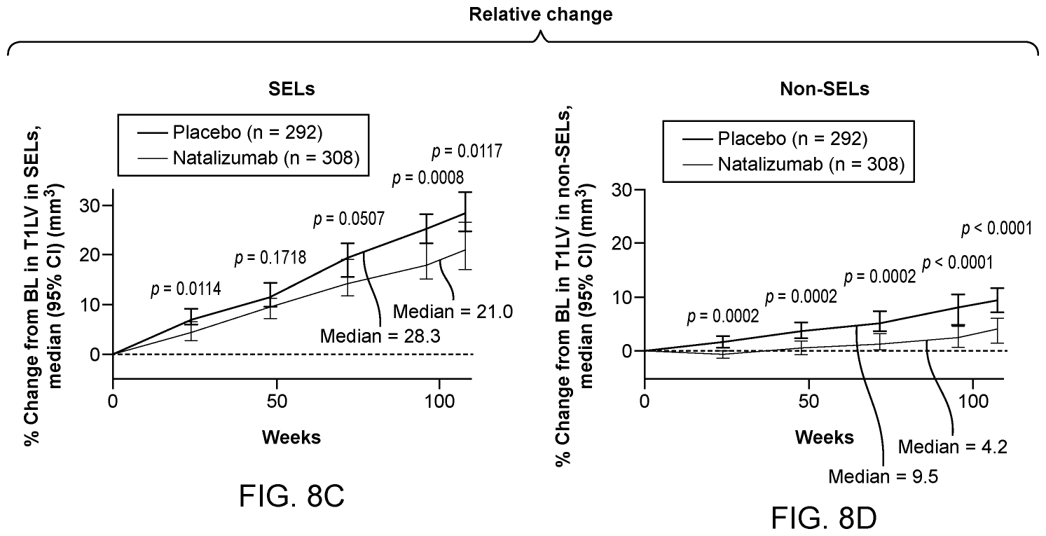

Brain tissue loss associated with CWMLA as measured by absolute and relative T1LV accumulation was lower in natalizumab—than placebo-treated patients within both SELs (FIG. 8A and C) and non-SELs (FIG. 8B and D).

### Discussion

SELs represent a subgroup of MS chronic white matter lesions with continual expansion and tissue destruction and predict clinical progression in progressive-onset MS. Elliott et al. Brain 2019; 142:2787-2799, Elliott et al. Mult Scler 2019; 25:1915-1925 and Elliott et al. AJNR Am J Neuroradiol 2020; 41:1584-1591. In SELs, there is an accumulation over time of T1LV and changes in imaging metrics reflective of gradual microstructural tissue alteration, including a decrease in magnetization transfer ratio and an increase in diffusion tensor imaging radial diffusivity. Elliott et al. AJNR Am J Neuroradiol 2020; 41:1584-1591. These findings suggest chronic demyelinating processes and continual axonal/neuronal destruction (Elliott et al. AJNR Am J Neuroradiol 2020; 41:1584-1591), which have been seen in pathology studies of chronic active lesions. Frischer et al. Ann Neurol 2015; 78:710-721 and Luchetti et al. Acta Neuropathol 2018; 135:511-528. However, it was recently reported that anti-inflammatory DMTs such as ocrelizumab (Elliott et al. Brain 2019; 142:2787-2799) and natalizumab (Preziosa et al. Mult Scler 2020: 1352458520969105) may have a modest effect on MRI measures of brain tissue loss in SELs.

In this study, SELs were identified in most patients with SPMS, and confirmed disability progression was associated with more severe CWMLA as measured by T1LV accumulation in SELs but also in non-SELs. These findings are consistent with previously reported findings in relapsing MS patients in the OPERA I and II dataset and primary progressive MS patients in the ORATORIO dataset. Elliott et al. Brain 2019; 142:2787-2799 and Elliott et al. Mult Scler 2019; 25:1915-1925). SPMS patients who progressed in a natural history setting had greater SEL prevalence and more severe CWMLA as measured by T1LV increase within SELs, non-SELs, and total baseline nonenhancing T2 lesions. In the absence of AWMLA in SPMS patients, SEL prevalence (as shown by number and volume of SELs) was reduced, though >70% of patients had ≥1 SEL. Importantly, the association of confirmed disability progression with more severe CWMLA in SPMS patients remained significant in the absence of AWMLA. Consistent findings were reported in placebo-treated primary progressive MS patients in the ORATORIO dataset, in whom AWMLA, CWMLA, and whole brain atrophy were measured and only CWMLA in SELs and non-SELs predicted confirmed disability progression over time. Elliott et al. Brain 2019; 142:2787-2799. This indicates that brain tissue loss associated with CWMLA may be an important driver of disability progression independent of AWMLA in MS.

Compared with placebo, natalizumab treatment significantly reduced the number and volume of SELs and the proportion of baseline nonenhancing T2 lesions identified as SELs. Natalizumab reduced CWMLA as measured by T1LV increase in both SELs and non-SELs. The significant association between AWMLA and SEL prevalence suggests that the effect of natalizumab on CWMLA in progressive MS patients could be related to its high efficacy in suppressing acute inflammation. Prior studies demonstrating the effects of natalizumab and depletion of CD20-expressing cells further support this finding. Kappos et al. JAMA Neurol 2020; 77:1132-1140, Montalban et al. N Engl J Med 2017; 376:209-220, Polman et al. N Engl J Med 2006; 354:899-910, Butzkueven et al. J Neurol Neurosurg Psychiatry 2014; 85:1190-1197, and Hauser et al. N Engl J Med 2017; 376:221-234. The effects of natalizumab on chronic active lesions have also previously been demonstrated in positron emission tomography studies measuring activated microglia. Kaunzner et al. Mult Scler Relat Disord 2017; 15:27-33 and Sucksdorff et al. Neurol Neuroimmunol Neuroinflamm 2019; 6:e574. In these studies, natalizumab decreased PK11195 uptake, reflective of activated microglia and macrophages, in nonenhancing lesions (Kaunzner et al. Mult Scler Relat Disord 2017; 15:27-33) and more specifically at the rim of chronic active lesions (Sucksdorff et al. Neurol Neuroimmunol Neuroinflamm 2019; 6:e574).

It is plausible that the smoldering inflammation that contributes to CWMLA and disability progression may be influenced by acute inflammation in MS. The recently published long-term brain MRI follow-up of primary progressive MS patients continuously treated with ocrelizumab over 6.5 years in the ORATORIO study (Wolinsky et al. Lancet Neurol 2020; 10:998-1009) demonstrated an annual increase in T1LV of approximately 3-6% (reaching a total increase of 37% from baseline at approximately 6 years), which may be attributable to CWMLA in SELs and non-SELs independent of AWMLA, as those patients were shown to be devoid of AWMLA from week 48 onward.

Without wishing to be bound by any one particular theory, the effects of natalizumab and/or ocrelizumab on CWMLA (in SELs and non-SELs) and/or disability progression in progressive forms of MS may be principally explained by their capacity to silence AWMLA. For example, an extended approach using Bayesian inference for a principal stratum estimand can be used to assess the treatment effect in subgroups characterized by AWMLA covariate thresholds as a postrandomization event occurrence. Magnusson et al. Stat Med 2019; 38(23): 4761-4771.

Efforts to further refine MS lesion histopathological terminology are ongoing (Kuhlmann et al. Acta Neuropathol 2017; 133:13-24), but understanding of the natural history of chronic active lesion phenotypes remains elusive, as their lifespan could begin decades before specimens become available. Dal-Bianco et al. Brain 2021; 144:833-847 doi.org/10.1093/brain/awaa436. Molecular studies of the lesion rim of chronic active or mixed active/inactive lesions show a predominance of M1-polarized macrophages and activated microglia. Jäckle et al. Brain 2020; 143:2073-2088. Innate and adaptive immune system interaction can bidirectionally influence polarization and perpetuate the disease process. Strachan et al. J Interferon Cytokine Res 2014; 34:615-622. Phase rims detected on susceptibility-weighted imaging are thought to represent an imaging biomarker of rims of iron-laden microglia/macrophages and have been linked to disease severity and brain atrophy. SELs (Kaunzner et al. Brain 2019; 142:133-145, Absinta et al. J Clin Invest 2016; 126:2597-2609, Dal-Bianco et al. Acta Neuropathol 2017; 133:25-42, and Absinta et al. JAMA Neurol 2019; 76:1474-1483) and phase rim lesions (PRLs) may both reflect chronic active lesions (Preziosa et al. Mult Scler 2020:1352458520969105). Recent work has demonstrated a partial concordance between SELs and PRLs, as a substantial proportion of PRLs do not appear to expand over time and some SELs appear to be devoid of phase rims entirely (Elliott et al. Neurology 2021; 96: (15 Supplement) 4101). As demonstrated herein, elucidating the complementarity of SEL, non-SEL, and PRL measures of CWMLA can expand the characterization of longitudinal tissue alteration properties within distinct MRI phenotypes of chronic active lesions.

In conclusion, CWMLA in SELs and non-SELs is an imaging biomarker of chronic active lesions and/or secondary axonal degeneration that reflects ongoing inflammation and is associated with clinical disability progression. We demonstrate that the presence of AWMLA, whether new Gd+ lesions or new T2-hyperintense lesions, is associated with a higher SEL prevalence in SPMS patients. This indirect evidence for the influence of AWMLA on CWMLA highlights the importance of limiting acute inflammation with highly effective disease-modifying therapies. However, even in the absence of AWMLA, the association between CWMLA and confirmed disability progression persists, albeit to a more limited extent, with the greater increase in T1w lesion volume in progressors than in nonprogressors seen primarily within non-SEL tissue of T2w nonenhancing lesions.

The onset of the effect of natalizumab on CWMLA was rapid in this study, with a statistically significant reduction in T1w lesion volume increase in SELs and non-SELs seen starting at 24 weeks of treatment. Taken together, these results highlight the need to continue to develop therapies that impact AWMLA but also more specifically target CWMLA, with the hope of blocking smoldering inflammation and neurodegenerative pathways, which may both contribute to CWMLA.

Patients with complete imaging datasets between baseline and week 108 (N=600) were analyzed for SEL prevalence (the number and volume of SELs), disability progression, AWMLA (assessed by gadolinium-enhancing lesions and new T2-hyperintense lesions), and CWMLA (assessed by T1-hypointense lesion volume increase within baseline T2-nonenhancing lesions identified as SELs and non-SELs).

Results: CWMLA in both SELs and non-SELs was greater in SPMS patients with confirmed disability progression than in those with no progression. In the complete absence of AWMLA at baseline and on study, SEL prevalence was significantly lower, while CWMLA within non-SELs remained associated with disability progression. Natalizumab decreased SEL prevalence and CWMLA in SELs and non-SELs compared with placebo.

### Example 2: MRI Characteristics of Phase Rim Lesions in Chronic and Recent Acute MS Lesions

Objective: To determine the prevalence of phase rim lesions (PRLs), their association with acute new T2-lesions, and to quantify normalized T1-weighted intensity (nT1), normalized magnetization transfer ratio (nMTR) and diffusion tensor imaging radial diffusivity (DTI-RD) in T2-lesions with (PRL+) and without (PRL−) phase rims, in a relapsing multiple sclerosis (RMS) population.

Background: Chronic active lesions are a subset of MS lesions thought to be represented by phase rim signals in susceptibility-weighted MR images.

Design/Methods: PRL data was collected at follow-up weeks 72 and 96 in a subset of RMS patients from the AFFINITY trial [NCT03222973] (N=44) using standardized 3-Tesla, 3D isotropic multi-echo, gradient echo MRI.

Results: 27 of 44 (61.4%) patients had at least 1 PRL at week 72 follow-up, and 11 of 44 (25.0%) had at least 4 PRLs. Approximately 10% of PRLs identified at week 72 derived from acute new lesions formed between baseline and week 72. Acute new T2 lesions that were PRL+ were larger at first detection (median size of 392 vs 52 mm 3) and had lower nMTR on average at detection and recovery stages compared to those acute new T2 lesions that were rimless. Chronic PRLs detected within T2 lesions pre-existing at baseline showed lower nMTR and higher RD at baseline. These lesions also showed a trend of decrease in nMTR and increase in RD from baseline to week 72 compared with chronic lesions without phase rims.

Conclusions: These data suggest that chronic T2 lesions with phase rims have more severe tissue injury than rimless chronic lesions. The minority of new T2 lesions that developed persistent phase rims were larger in size and also associated with more severe acute tissue damage as measured by nMTR decrease.

### Example 3: MRI Characteristics of Chronic MS Lesions by Phase Rim Detection and/or Slowly Expanding Properties

Objective: To evaluate the co-localization of phase rim lesions (PRLs) and slowly expanding lesions (SELs) and compare normalized magnetization transfer ratio (nMTR) and diffusion tensor imaging radial diffusivity (DTI-RD) in PRLs and SELs in patients with relapsing multiple sclerosis (RMS).

Background: PRLs, as detected on susceptibility-weighted phase images, have been associated with chronic active MS lesions (Absinta et al. J Clin Invest 2016; 126:2597-2609). SELs have been posited as a marker of chronic active MS lesions that can be assessed using only conventional MRI sequences (Elliot et al. Multiple Sclerosis Journal 2018; DOI: 10.1177/1352458518814117). These lesions types may be relevant biomarkers, as SELs have been associated with ongoing tissue damage within lesions that is predictive of disability progression in progressive-onset MS (Elliott et al. Brain 2019; 142:2787-2799, and Elliott et al. AJNR Am J Neuroradiol 2020; 41(9): 1584-1591), and patients with ≥4 PRLs demonstrated faster accumulation of disability Absinta et al. JAMA Neurol 2019; 76:1474-1483). However, the degree of overlap of SELs with PRLs is unknown.

Design/Methods: Study Design: Brain MRI data were acquired in AFFINITY [NCT03222973], a phase II trial of opicinumab in relapsing MS with an initial blinded, placebo controlled portion followed by an open label extension study. Patients stable on disease modifying therapies (DMTs) (interferon, natalizumab, or dimethyl fumarate) were randomized to receive 750 mg opicinumab every 4 weeks of placebo in addition to their background DMT.

Imaging: The imaging protocol included T1-weighted scans pre-/post gadolinium, T2-weighted FLAIR, PD-weighted and T2-weighted spin echo images, 2 spoiled gradient-recalled echo images with/without an MT pulse for calculating magnetization transfer ratio (MTR), and diffusion-weighted imaging using 32 directions. In Part 2 only, the protocol also included susceptibility weighted imaging (SWI) using standardized 3T Siemens 3D isotropic multi-echo spoiled gradient T2*. PRLs were detected from SWI phase images at Week 72 or Part 2/Day1. SELs were detected as areas of Part 1 baseline T2 lesions that showed constant and concentric expansion from baseline to Week 72, using longitudinal T1- and T2-weighted images.

Results: 41 of the patients who participated in the advanced MRI sub-study of AFFINITY Part 2 had SWI available at week 72. Patient characteristics are shown in Table 5.

TABLE 5
Baseline Characteristics (n = 41)

| | |
|---|---|
| Age, mean (sd), years | 38.7 (9.49) |
| Disease duration, mean (sd), years | 6.9 (5.16) |
| EDSS, median (IQR) | 2.5 (2.5) |
| T2 lesion volume, mean (sd), ml | 8.8 (10.61) |
| Normalized brain volume, mean (sd), ml | 1457.3 (117.7) |
| Proportion with Gd-enhancing lesions | 0.15 |

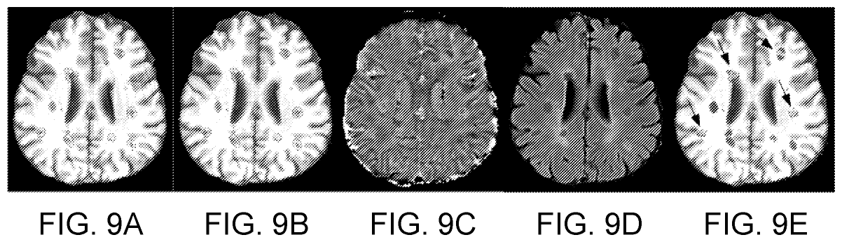

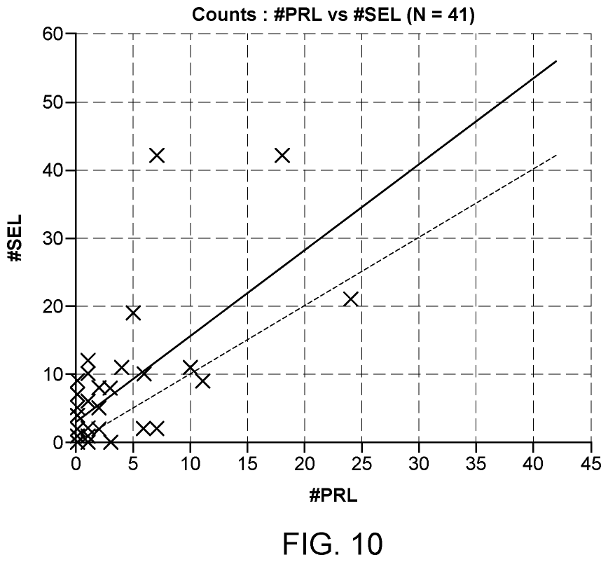

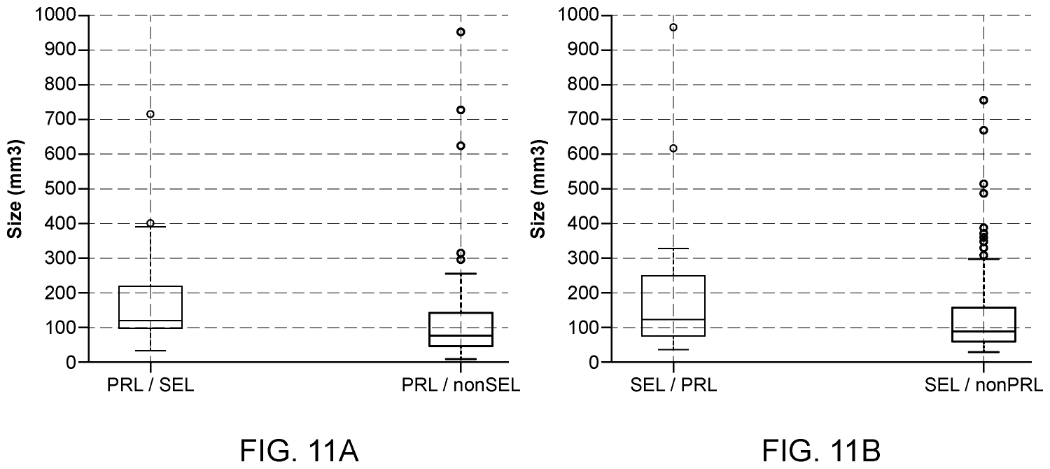

Cumulatively, over the 41 patients analyzed, more than twice as many SELs (267) were detected as PRLs (119). Most SELs and PRLs were non-overlapping (FIG. 9e). 39.5% of PRLs ( 47/119) co-localized with SELs while 17.2% ( 46/267) of SELs co-localized with PRLs. Moderate correlation of SEL and PRL counts across patients was observed (r=0.67) (FIG. 10). Lesions with SEL/PRL co-localizations were larger than SEL-only or PRL-only lesions. PRLs colocalizing with SELs were larger in size than those that did not (FIGS. 11A-11B and Table 6).

TABLE 6
Comparison of Lesion Sizes

| | PRL | PRL non | SEL w | SEL non |
|---|---|---|---|---|
| | w/SEL | SEL | PRL | PRL |
| Mean (sd) size (mm³) | 171.7 | 124.5 | 327.8 | 134.0 |
| | (124.5) | (156.6) | (629.5) | (168.1) |
| Median Size (mm³) | 118.9 | 75.0 | 120.4 | 87.8 |

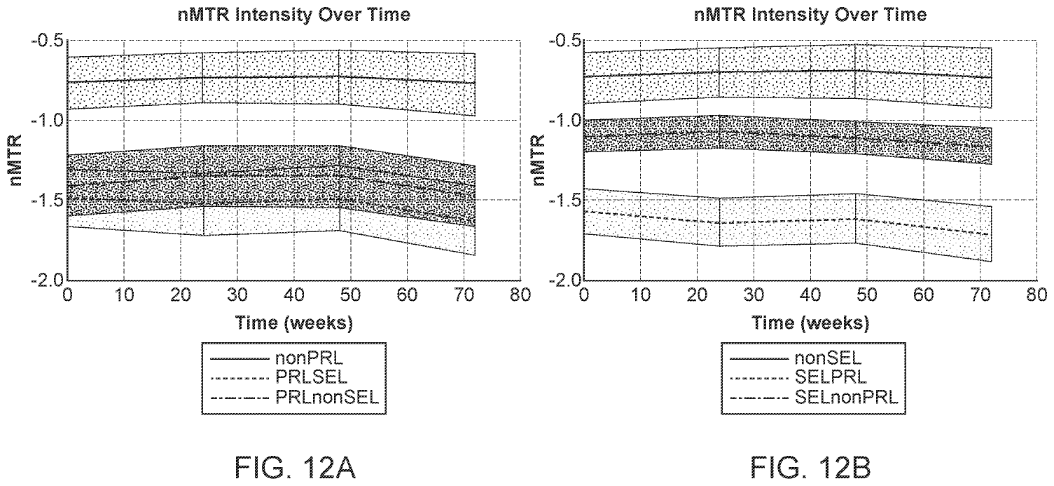

Chronic lesions that were detected as both PRL+/SEL+ had lowest normalized magnetization transfer ratio (nMTR) (FIGS. 12A-12B) and higher diffusion tensor imaging radial diffusivity

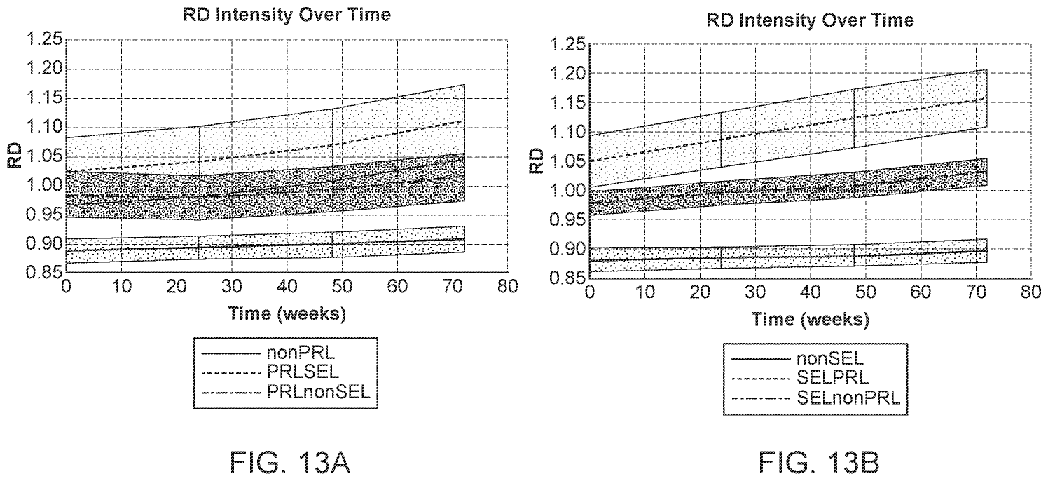

(DTI-RD), compared to PRLs with no SEL properties and SELs with no associated PRLs (FIGS. 13A-13B).

Conclusions: White matter lesions defined as SELs and PRLs show only partial correspondence and only a minority of SELs are associated with phase rims and vice versa. SELs and PRLs that co-localized may represent the most severe subset of chronic active white matter lesions. Ongoing investigations of SELs and PRLs may help to clarify MRI lesion subtypes and lead to more sensitive markers of MS disease progression.

### Example 4: MRI Characteristics of Chronic MS Lesions by Phase Rim Detection and/or Slowly Expanding Properties

Objective: To identify a low-dimensional signature of radiomics textural biomarkers discriminative of SEL versus non-SEL MS lesion activity, and to develop a ML-based classifier discriminating SELs from non-SELs in chronic non-enhancing white matter MS lesions, from cross-sectional T1- and T2-weighted brain MRI.

Background: Chronic active lesions are thought to play a role in the progressive biology of MS through insidious damage to myelin and axons by chronic inflammatory processes occurring within pre-existing MS lesions (Luchetti et al. Acta Neuropathol. 2018; 135(4):511-528). Some chronic active lesions can be detected as slowly expanding lesions (SELs) identifiable on MRI as contiguous regions of existing T2 lesions showing gradual concentric expansion (Elliott C, et al. Mult Scler. 2019; 25(14): 1915-1925). Machine learning and texture analysis techniques may allow for the discrimination of MS lesion subtypes on conventional MRI imaging. In multiple sclerosis, disease activity is traditionally classified into two forms: relapsing MS or progressive MS. While conventional MRI provides reliable bio-markers of acute disease activity associated with the relapsing form of the disease, there exists comparatively fewer established biomarkers for the detection of tissue states characterizing the progressive phase of MS.

In this context, it is generally believed that chronic active lesions, which are pathologically characterized by a rim of activated microglia and macrophages, may play an important role in the progressive biology of MS. Across the class of chronic active lesions, some can be detected as SELs. These lesions are identifiable on T1-weighted MRI as contiguous regions of existing T2 lesions showing gradual concentric expansion. Current methods for SEL detection thus rely on at least 3 longitudinal scans acquired over a period of 1 to 2 years of follow-up. The requirement for longitudinal data results in delayed quantification of SEL activity, which is in part relevant in the context of clinical trials for DMTs targeting progressive MS. Therefore, the aim of this Example is to detect SELs in the cross-sectional setting. The algorithm presented in this Example leverages techniques of image processing from the fields of radiomics analysis combined with machine learning. As such, the solution is designed to identify a signature of textural biomarkers associated with SEL activity, and to use this signature to discriminate SELs from non-SELs, within the bounds of white matter hyperintensities.

Design/Methods: T1-weighted and T2-weighted MRIs were retrospectively analysed (ADVANCE—1512 patients with relapse-remitting MS; ASCEND—886 patients with secondary progressive MS; SYNERGY—419 patients with relapse remitting MS/secondary progressive MS). Ground truth SELs were detected in each baseline scan using a Jacobian integration-based method (Elliott C, et al. Mult Scler. 2019; 25(14): 1915-1925) leveraging longitudinal MRI data spanning 1 to 2 years of follow-up. Briefly, ground truth SELs were detected in each baseline scan using a Jacobian-based method analysing the evolution in T1 intensity across a series of longitudinal scans.

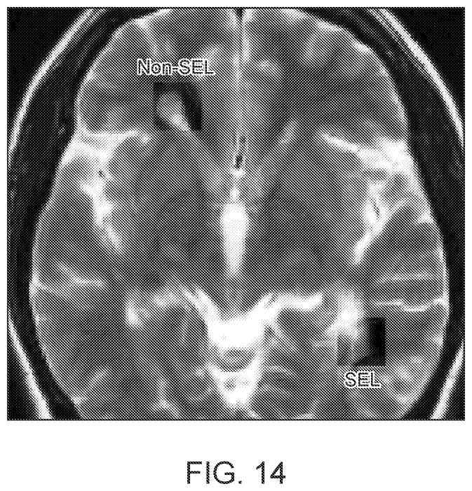

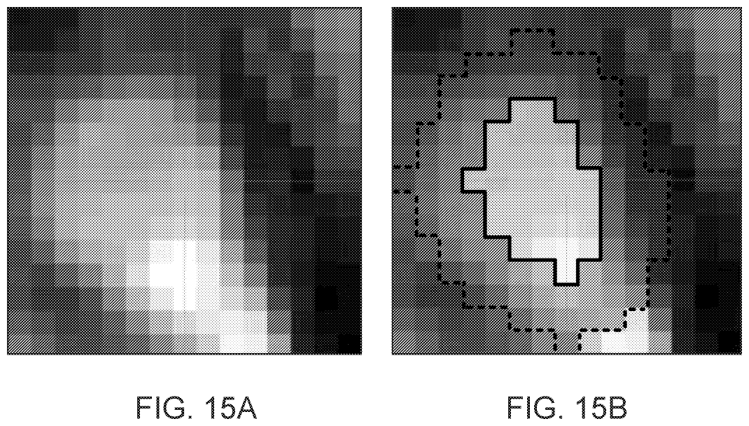

Cubic patches of 15×15×15 mm were extracted from the SEL and non-SEL tissue of each baseline scan (FIG. 14). Specifically, cubic patches of 15×15×15 mm were sampled randomly from SELs and non-SELs across all available baseline scans. For each patch, texture-based radiomic features were extracted separately from the core and periphery of the patch, as shown at FIG. 15B in green and red, respectively. In more details, referring to FIG. 15B, the “core” region contains all lesion voxels located less than 4 mm away from the central voxel of the patch. The “periphery” region contains voxels within a 3-mm margin outside the edge of the core region. For each patch, a set of 372 radiomic features was extracted from T1- and T2-weighted MRI data.

The feature selection algorithm evaluated the discriminative value of each feature. Using selected features, a pool of ML models were benchmarked. The 5 top-performing models were ensembled using a stacking strategy. A recursive loop further eliminated noninformative features. Briefly, patients from ADVANCE were split 80:20 into training and validation sets, respectively. The training set was used as input to a feature selection and ensemble classification pipeline. The feature selection pipeline evaluated the predictive value of each one of the 372 features, using an ensemble of correlation tests evaluating the association of each feature with the label of the patch, both in the univariate and in the multivariate setting. This approach produced a ranking of the radiomic features from most useful to least useful. In the input space defined by the 50 most useful features, a pool of standard machine learning models were benchmarked via 10-fold cross-validation using patient-level splits. The 5 top-performing models were combined under a stacking ensemble strategy.

The dimensionality of the radiomics feature space was further compressed by recursively eliminating non-informative features one-by-one. This led to a compact signature of only 15 radiomics variables, along with an optimal classifier able to interpret this signature to discriminate SELs from non-SELs. This optimal classifier was subsequently tested on both the validation set of patches from ADVANCE patients, and the independent sets of patches from the ASCEND and SYNERGY patients.

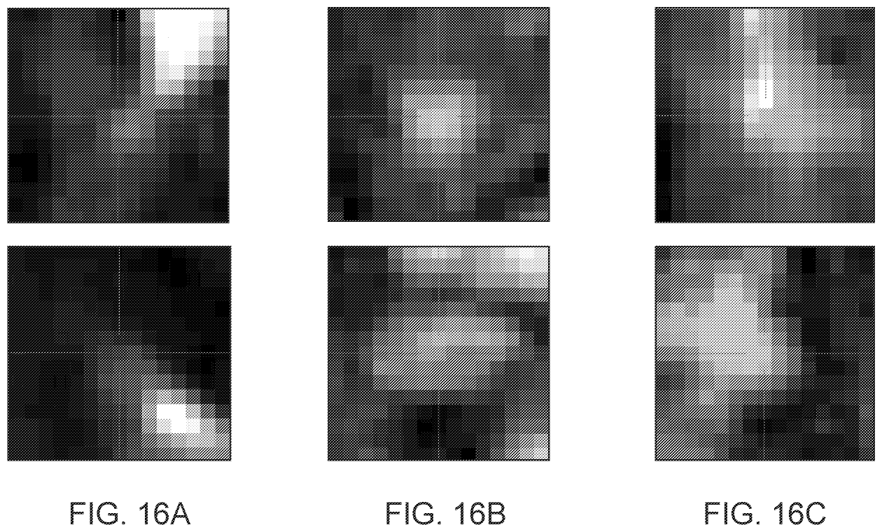

We additionally trained a new classifier to discriminate SELs from non-SELs under strict matching of the volume of lesion contained in SEL versus non-SEL patches (FIGS. 16A-16C). This ensures that volume-independent textural biomarkers are detected.

This entire pipeline was applied twice: once under random sampling of SEL and non-SEL patches, and a second time under strict matching of the volume of lesion found in SEL versus non-SEL patches. This volume matching experiment allows us to evaluate our ability to discriminate SELs from non-SELs based purely on textural biomarkers and independently of geometry.

To clarify, although both experiments were restricted to radiomic features quantifying texture, it is important to recognize that some of these features, such as the entropy and the energy, are volume-confounded. Therefore, a strict volume matching paradigm can ensure a volume-independent classification.

Results: The 15 radiomic features selected via our recursive elimination pipeline define a compact signature discriminative of SEL versus non-SEL activity. This signature primarily contains information from T1-weighted MRI signals in the core of the patch. We observed that first-order statistics including the mean, median and 90th percentile of T1 intensities in the core of the patch were identified as relevant, which is consistent with prior studies reporting that SELs exhibit a higher degree of T1 hypo-intensity relative to non-SELs at baseline. Radiomics signature of the 15 features included 10 from T1-weighted MRI, 5 from T2-weighted MRI, 8 from the “core” of patches, and 7 from the “periphery” of patches. Prevalence of each of the 15 radiomic features selected for discriminating SEL from non-SEL patches is shown in FIG. 17.

When discriminating SEL from non-SEL patches, our optimal classification model achieved 67% balanced accuracy on the ADVANCE validation set, 66% on the ASCEND test set and 69% on the SYNERGY test set. Importantly, we achieved a high sensitivity and a low specificity when testing on the SPMS ASCEND population, while in contrast we observed a low sensitivity and a high specificity when testing on the SYNERGY population (Table 7).

TABLE 7
Summary statistics for the performance of the classification
model for patch-level SEL versus non-SEL discrimination
on the validation and independent testing sets

| | Balanced | | | | ROC |
|---|---|---|---|---|---|
| Dataset | accuracy | Precision | Sensitivity | Specificity | AUC |
| ADVANCE | 67% | 66% | 70% | 64% | 73% |
| (validation) | | | | | |
| ASCEND | 66% | 63% | 76% | 56% | 71% |
| (testing) | | | | | |
| SYNERGY | 69% | 67% | 68% | 71% | 74% |
| (testing) | | | | | |

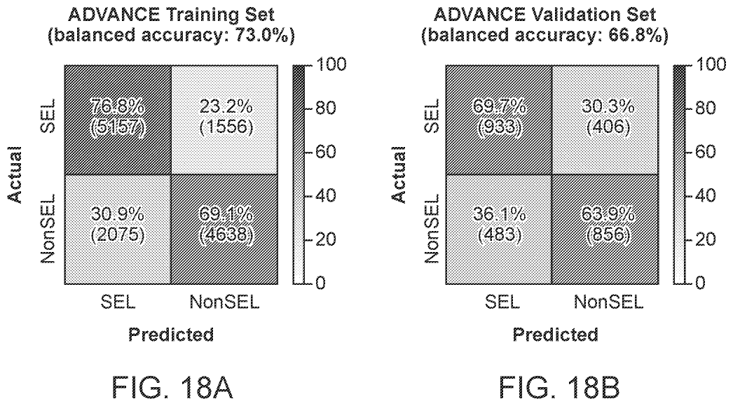

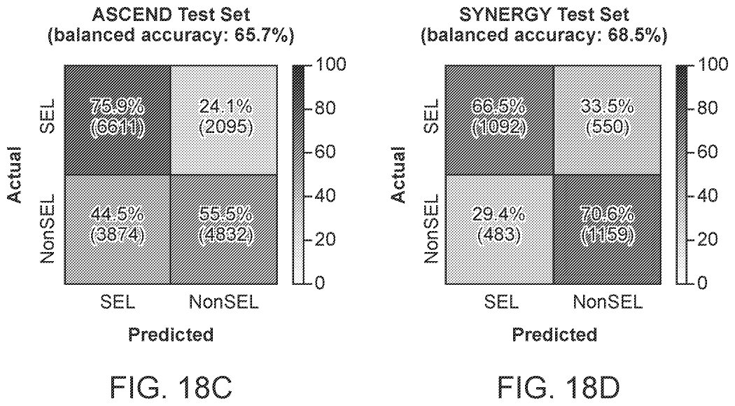

FIGS. 18A-18D are confusion matrices showing the performance of the classification model for patch-level SEL versus non-SEL discrimination on the training, validation, and independent testing sets.

In the volume-balanced setting, the resulting ensemble model achieved 62% balanced accuracy on the ADVANCE validation set, as well as on the SYNERGY and ASCEND test sets (Table 8). The small drop in performance reported in this volume-balanced experiment relative to the results reported in the previous table indicate that our initial experiment did capture volumetric differences between SELs and non-SELs, beyond textural information.

TABLE 8
Summary statistics for the performance of the classification model
for patch-level SEL versus non-SEL discrimination on the validation
and independent testing sets for volume-matched patches.

| | Balanced | | | | ROC |
|---|---|---|---|---|---|
| Dataset | accuracy | Precision | Sensitivity | Specificity | AUC |
| ADVANCE | 62% | 62% | 63% | 61% | 66% |
| (validation) | | | | | |
| ASCEND | 62% | 61% | 68% | 57% | 66% |
| (testing) | | | | | |
| SYNERGY | 62% | 63% | 55% | 68% | 67% |
| (testing) | | | | | |

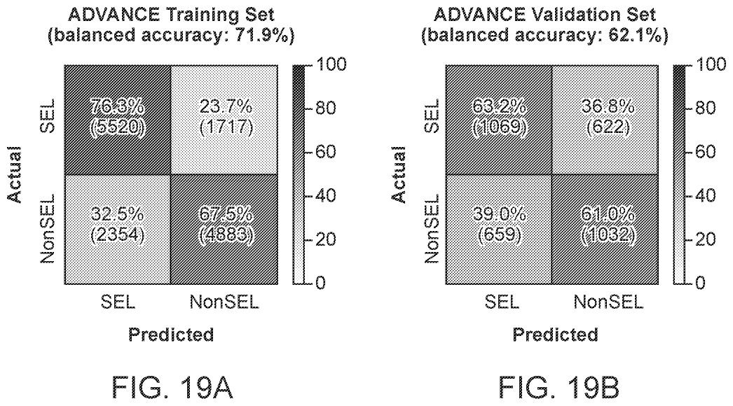

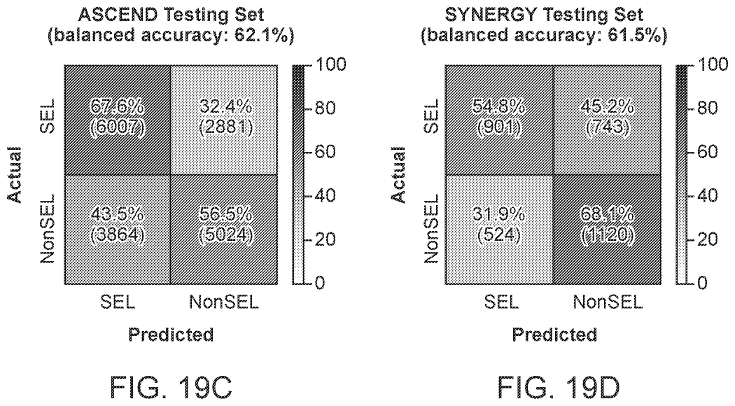

FIGS. 19A-19D are confusion matrices showing the performance of the classification model for patch-level SEL versus non-SEL discrimination on the training, validation, and independent testing sets for volume-matched patches.

Conclusions: A machine learning classifier was developed that is able to discriminate SELs from non-SELs using single-timepoint non-contrast conventional T1- and T2-weighted MRI, with classification accuracy ranging from 66% to 69% under random patch sampling, versus 62% under strict lesion volume matching. This indicates that SELs may express patterns of conventional non-enhancing MRI signals detectable using ML techniques in the cross-sectional setting. The single-timepoint detection of SELs may alleviate the need for longitudinal analysis and enable baseline quantification of chronic MS lesion subtypes. Applications of the algorithm could include population enrichment in clinical trials and improved patient prognostication in the clinical setting. Future work can incorporate chronic active lesions detectable by paramagnetic rim identification into the classification algorithm.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

What is claimed is:

1. A method of treating Radiologically Isolated Syndrome in a patient in need thereof comprising administering a therapeutically effective amount of a disease-modifying antibody therapy to said patient, wherein said patient has at least one phase rim lesion (PRL) in at least one susceptibility-weighted magnetic resonance image (MRI), or the patient has at least one slowly expanding lesion (SEL) in at least one T1-weighted/T2-weighted MRI.

2. A method of reducing and/or treating chronic active white matter lesions in an early-stage and/or asymptomatic MS patient comprising administering a therapeutically effective amount of a disease-modifying antibody therapy to said patient, wherein said patient has at least one PRL in at least one susceptibility-weighted MRI, or the patient has at least one SEL in at least one T1-weighted/T2-weighted MRI.

3. The method according to claim 1 or 2, wherein treatment with disease-modifying antibody therapy is initiated when at least 1%, 5%, 10%, 15%, 20%, 25%, 30%, 35%, 40%, 45%, 50%, 55%, 60%, 65%, 70%, or 75% of the patient's total T2 hyperintense lesion volume and/or number is identified as PRL.

4. The method according to claim 1 or 2, wherein treatment with disease-modifying antibody therapy is initiated when at least 1%, 5%, 10%, 15%, 20%, 25%, 30%, 35%, 40%, 45%, 50%, 55%, 60%, 65%, 70%, or 75% of the patient's total T2 hyperintense lesion volume and/or number is identified as SEL.

5. The method of claim 1 or 2, wherein the patient has at least one SEL that co-localizes with at least one PRL, or at least one PRL that co-localizes with at least one SEL, optionally wherein said at least one SEL is detected using single time-point non-contrast T1- and T2-weighted MRI.

6. The method according to claim 5, wherein treatment with disease-modifying antibody therapy is initiated when at least 5%, 10%, 15%, 20%, 25%, 30%, 35%, 40%, 45%, 50%, 55%, 60%, 65%, 70%, or 75% of the patient's SELs co-localize with their PRLs, and/or when at least 5%, 10%, 15%, 20%, 25%, 30%, 35%, 40%, 45%, 50%, 55%, 60%, 65%, 70%, or 75% of the patient's PRLs co-localize with their SELs.

7. The method of any preceding claim, wherein the SEL is detected using a machine-learning based classifier that discriminates acute from chronic MS lesions and/or SEL from non-SEL using single time-point non-contrast T1- and T2-weighted MRI.

8. The method of any preceding claim, wherein said disease-modifying antibody therapy is selected from natalizumab, BIIB107 and ocrelizumab.

9. The method of any preceding claim, wherein the disease-modifying antibody therapy is natalizumab.

10. The method according to claim 9, wherein natalizumab is administered in a biphasic dosing regimen, wherein the biphasic regimen comprises an induction phase comprising administration of natalizumab once a month for about 10 to about 14 months, followed by a chronic phase comprising administration of natalizumab once every 5, 6, 7 or 8 weeks.

11. The method according to claim 10, wherein at least one phase of the biphasic protocol comprises subcutaneous (SC) administration.

12. The method according to claim 10, wherein both phases of the biphasic protocol comprise SC administration.

13. A method for reducing and/or treating chronic lesion activity in an asymptomatic and/or early-stage MS patient (e.g. having no relapse events) comprising a) identifying at least one phase rim lesion (PRL) in at least one susceptibility-weighted magnetic resonance image from a patient known or suspected of having chronic lesion activity, b) identifying at least one slowly-expanding lesion (SEL) in at least one T1-weighted/T2-weighted MRI from said patient; c) determining if the at least one PRL co-localizes with the at least one SEL in said patient, and/or vice-versa, and d) in the event of co-localization initiating treatment with a disease-modifying antibody therapy.

14. The method of claim 13, wherein the at least one SEL is detected using a machine-learning based classifier that discriminate acutes from chronic MS lesions and/or SEL from non-SEL using single time-point non-contrast T1- and T2-weighted MRI.

15. The method according claim 13 or claim 14, wherein treatment with said disease-modifying antibody therapy is initiated when at least 5%, 10%, 15%, 20%, 25%, 30%, 35%, 40%, 45%, 50%, 55%, 60%, 65%, 70%, or 75% of the patient's SELs co-localize with their PRLs, and/or when at least 5%, 10%, 15%, 20%, 25%, 30%, 35%, 40%, 45%, 50%, 55%, 60%, 65%, 70%, or 75% of the patient's PRLs co-localize with their SELs.

16. The method according to any one of claims 13 to 15, wherein the disease-modifying antibody therapy is selected from natalizumab, BIIB107 and ocrelizumab.

17. The method according to any one of claims 13 to 15, wherein the disease-modifying antibody therapy is an anti-VLA-4 antibody, e.g. natalizumab or BIIB107.

18. The method according to claim 17, wherein the anti-VLA-4 antibody is natalizumab, and the method further comprises administering to said patient a therapeutically effective amount of natalizumab in a biphasic dosing regimen, wherein the biphasic regimen comprises an induction phase comprising administration of the anti-VLA-4 antibody once every 2 weeks, once every 4 weeks, once every 30 days, or once a month for at least 6 months, for at least 8 months, for at least 10 months, or for at least 12 months, followed by a chronic phase comprising administration of natalizumab once every 5 to 10 weeks.

19. The method according to claim 18, wherein the chronic phase comprises administration of natalizumab once every 5 weeks, once every 6 weeks, once every 7 weeks, or once every 8 weeks.
